(19) United States
(12) Patent Application Publication
(10) Pub. No.: US 2010/0316613 A1
(43) Pub. Date: Dec. 16, 2010

(54) FEEDER CELL-FREE CULTURE MEDIUM AND SYSTEM

(76) Inventors: Zee Upton, Queensland (AU); David Leavesley, Queensland (AU); Sean Dennis Richards, Queensland (AU); Luke Bryant Cormack, Queensland (AU); Damien Harkin, Queensland (AU)

Correspondence Address: TAROLLI, SUNDHEIM, COVELL & TUMMINO L.L.P., 1300 EAST NINTH STREET, SUITE 1700, CLEVELAND, OH, 44114, US

(21) Appl. No.: 12/676,341

(22) PCT Filed: Sep. 3, 2008
(86) PCT No.: PCT/AU08/01308

§ 371 Date: Jun. 25, 2010

(30) Foreign Application Priority Data
Sep. 4, 2007 (AU) 2007904793
Feb. 27, 2008 (AU) 2008900955

(51) Int. Cl. A61K 35/12; C12N 5/02; C12N 5/0735; C12M 3/00; A61K 35/36; A61P 17/02

(52) U.S. Cl. 424/93.7; 435/406; 435/405; 435/289.1; 435/366

(57) ABSTRACT

A cell culture medium and system are provided which eliminates or at least reduces the need for feeder cells. The cell culture medium comprises one or more factors that are normally secreted and/or produced by a feeder cell and a synthetic chimeric protein comprising IGF-I and a portion of vitronectin. The cell culture medium is particularly suitable for propagating human embryonic stem cells and keratinocytes. This invention also relates to compositions and methods which utilize the cells cultured in the cell culture medium of the invention.

### FIELD OF THE INVENTION

THIS INVENTION relates to cell culture. More particularly, this invention relates to a medium, system and method for a feeder cell independent cell culture system.

### BACKGROUND TO THE INVENTION

Human embryonic stem (hES) cells are derived from the inner cell mass (ICM) of a blastocyst, which is an early stage embryo approximately 4 to 5 days old. The hES cell is a pluripotent cell type that can give rise to the three primary germ layers, namely ectoderm, endoderm and mesoderm [1, 2]. In other words, these cells can develop into more than 200 cell types of the adult body when given the necessary stimulation for differentiation. Alternatively, when given no stimulation for differentiation, these cells will self renew giving rise to pluripotent daughter cells.

In light of this, it is thought that the pluripotential behaviour of hES cells can be manipulated to more efficiently generate cells and tissues for therapeutic applications: for example, Parkinson's disease [3], diabetes [4], or spinal cord injuries [5]. However, these potential applications extend to more than just generation of tissues for transplantation. Recently hES cells have been manipulated to form specific tissue types for testing new drugs and chemicals [6]. Nevertheless, despite these advances, hES cells will not be therapeutically viable until safe culture methodologies are established.

The first successful derivation of hES cells was achieved in 1998 [1]. Thomson et al. (1998) discovered that hES cells could be successfully propagated using a mitotically inactivated feeder layer and foetal bovine serum (FBS). However, the use of xenogeneic products, such as human or animal serum and mouse fibroblasts, can lead to the introduction of contaminating products, such as Bovine Spongiform Encephalopathy, to the culture system [7, 8]. More recently, the addition of these animal components has also been demonstrated to introduce immunogenic agents (eg. N-glycolylneuraminic acid, Neu5Ac) (Martinet al. 2005; Heiskanen et al. 2007) suggesting that the cells grown in these conditions can be phenotypically manipulated by their micro-environment. Clearly, improved hES cell culture methodologies need to be developed, whilst at the same time providing the necessary conditions for hES cell in-vitro expansion.

In view of this many researchers have investigated the use of human feeder cells including, human foreskin fibroblasts [9, 10] and human adult marrow cells [11]. Both of these have been demonstrated to support hES cell growth, thereby removing the risk of contamination from animal derived feeder cells. However, studies have revealed that greater rates of differentiation and abnormal karyotypes occur after prolonged propagation [12, 9]. For example, some hES cells subjected to cytogenetic analysis display aneuploidy [12], including the gain of chromosome 17q [13] and trisomy 20 [14].

To address this many researchers have been attempting to develop hES cell culture conditions which are completely free of animal products. In particular, Xu et al. (2005) has identified basic fibroblast growth factor (bFGF) signalling to be critically important for hES cell self-renewal [15], whereas other researchers have postulated that modulating the transforming growth factor (TGF)-β signalling pathway is necessary for preventing differentiation by default [16]. More recently, Ludwig et al. (2006) demonstrated a successful feeder-free culture of hES cells using a complex mixture of proteins and large quantities of purified human serum albumin [17]. However, this study revealed a 47 XXY karyotype when the hES cells were cultured for several months. Whilst this was a significant step forward, it is clear that hES cells still require feeder cells for their successful propagation. Interestingly, Xu et al. (2001) demonstrated that conditioned medium (CM) from mouse embryonic fibroblast (MEF) cells can support hES cell growth up to 130 population doublings, whilst still maintaining their normal karyotype [18].

Another cell type that relies on mouse fibroblasts feeder cells for their establishment and expansion are primary human keratinocyte cells. Indeed, many of the culture techniques used for the propagation of hES cells i.e. serum and feeder cells, are analogous to those used in keratinocyte culture. It has been demonstrated that primary keratinocytes have a reliance on the mouse fibroblast feeder cells for their undifferentiated expansion in-vitro (Dawson et al. 2006).

To date it is not yet understood what function the MEFs have in hES cell culture. However, it has been demonstrated that these feeder cells supply a range of proteins which may be vital for maintaining the hES cells, and perhaps also keratinocyte cells, in an undifferentiated state.

### SUMMARY OF THE INVENTION

Existing cell culture systems that rely upon a mitotically inactive feeder layer of cells to supply growth and conditioning factors for propagation and/or proliferation of cells have severe potential drawbacks in therapeutic applications. More particularly, use of xenogeneic products may introduce contaminating and infectious agents such as BSE and HIV.

Therefore, the present inventors have identified a requirement for a new and improved a cell culture system which obviates or at least reduces the need for feeder cells. Moreover, the inventors have surprisingly found that a synthetic chimeric protein comprising an IGF-I amino acid sequence and amino acid residues 1 to 64 of mature vitronectin displays higher activity in the cell culture medium, and in particular is able to stimulate cell migration and/or proliferation to high levels.

In one broad form, the invention relates to a serum-free non-conditioned cell culture medium comprising one or more isolated feeder cell-replacement factors for use as a substitute or replacement for feeder cells. It is envisaged that the one or more isolated feeder cell-replacement factors can be any protein, or a biologically active fragment thereof, which is normally secreted and/or produced by a feeder cell so as to facilitate growth of a feeder-dependent cell.

In a first aspect, the invention provides a cell culture medium, comprising:

(i) a synthetic chimeric protein comprising an insulin-like growth factor (IGF) amino acid sequence and a vitronectin (VN) amino acid sequence;

(ii) one or more isolated feeder cell-replacement factors selected from the group consisting of human growth hormone (hGH), bone morphogenic protein 15 (BMP-15), growth differentiation factor 9 (GDF-9), megakaryocyte colony-stimulating factor, secreted frizzled-related protein 2, Wnt-2b, Wnt-12, growth inhibitory factor, fetuin, human serum albumin (HSA), hepatocyte growth factor (HGF), transforming growth factor-α (TGF-α), transforming growth factor-β (TGF-β), nerve growth factor, platelet derived growth factor-β (PDGF-β), PC-derived growth factor (progranulin), interleukin (IL)-1, IL-2, IL-4, IL-6, IL-8, IL-10, IL-13 and Activin-A; and

(iii) an absence of serum or a substantially reduced amount of serum which in the absence of an IGF would not support cell growth.

Preferably, the one or more isolated feeder cell-replacement factors are selected from the group consisting of hGH, BMP-15, GDP-9, megakaryocyte colony-stimulating factor, secreted frizzled-related protein 2, Wnt-2b, Wnt-12, growth inhibitory factor and Activin-A.

Even more preferably, the one or more isolated feeder cell-replacement factors is Activin-A.

In preferred embodiments, the cell culture medium further comprises one or more additional biologically active proteins selected from the group consisting of basic fibroblast growth factor (bFGF), epidermal growth factor (EGF), IGF-I, IGF-II and a laminin.

In more preferred embodiments, the one or more additional biologically active proteins are selected from bFGF and a laminin.

Preferably, the IGF amino acid sequence is an IGF-I amino acid sequence or an IGF-II amino acid sequence.

More preferably, the IGF amino acid sequence is an IGF-I amino acid sequence.

In a preferred embodiment, the VN amino acid sequence is amino acid residues 1 to 64 of mature vitronectin.

Preferably, the synthetic chimeric protein further comprises a linker sequence of one or more glycine residues and in particularly preferred embodiments, said linker sequences further comprise one or more serine residues.

More preferably, the linker sequence is (Gly₄Ser)₄

In another preferred embodiment, the cell culture medium further comprises an isolated IGF-containing complex wherein the IGF is selected from IGF-I and IGF-II.

In another preferred embodiment where the isolated IGF-containing complex comprises IGF-I, the cell culture medium further comprises an insulin-like growth factor binding protein (IGFBP) and VN.

In yet another preferred embodiment where the IGF present in the isolated IGF-containing complex is IGF-II, the cell culture medium further comprises VN.

Preferably, the or each feeder cell-replacement factor has a final concentration of between about 0.1 ng/ml and 50 μg/ml.

More preferably, the or each feeder cell-replacement factor has a final concentration of between about 5 ng/ml and 1500 ng/ml.

Even more preferably, the or each feeder cell-replacement factor has a final concentration of between about 25 ng/ml and 1000 ng/ml.

Yet more preferably, the or each feeder cell-replacement factor has a final concentration of between about 150 ng/ml and 600 ng/ml.

Yet even more preferably, the or each feeder cell-replacement factor has a final concentration of between about 250 ng/ml and 400 ng/ml.

Suitably, the cell culture medium is for use in culturing a feeder-dependent cell.

It is readily appreciated that the feeder-dependent cell is any cell which requires a feeder cell for propagation. Non-limiting examples include mouse and human embryonic stem cells, human embryonic germ cells, human embryonic carcinomas and keratinocytes.

Preferably, the feeder-dependent cell is selected from human embryonic stem cells and keratinocytes.

In a second aspect, the invention provides an embryonic cell culture medium comprising between about 250 ng/ml and 1000 ng/ml of a synthetic chimeric protein comprising an IGF amino acid sequence and a VN amino acid sequence, between about 50 ng/ml and 100 ng/ml of bFGF, between about 25 ng/ml and 50 ng/ml of Activin-A and between about 10 μg/ml and 50 μg/ml of a laminin.

Preferably, the embryonic stem cell culture medium comprises about 1000 ng/ml of the synthetic chimeric protein, about 100 ng/ml of bFGF, about 35 ng/ml Activin-A and about 40 μg/ml of a laminin.

Preferably, the IGF amino acid sequence is an IGF-I amino acid sequence or an IGF-II amino acid sequence.

More preferably, the IGF amino acid sequence is an IGF-I amino acid sequence.

In a preferred embodiment, the VN amino acid sequence is amino acid residues 1 to 64 of mature vitronectin.

In a third aspect, the invention provides a cell culture system comprising a culture vessel and the cell culture medium of the first aspect or the embryonic stem cell culture medium of the second aspect.

In a fourth aspect, the invention provides a method of cell culture including the step of culturing one or more cells in the cell culture medium of the first aspect, the embryonic stem cell culture medium of the second aspect and/or the cell culture system of the third aspect.

Preferably, the one or more cells are feeder-dependent cell types.

More preferably, the one or more cells are hES cells or keratinocytes.

In a fifth aspect, the invention provides a pharmaceutical composition comprising one or more cells produced according to the method of the fourth aspect, together with a pharmaceutically acceptable carrier, diluent or exicipient.

In a preferred embodiment, the pharmaceutical composition comprises one or more cells selected from the group consisting of hES cells, keratinocytes and keratinocyte progenitor cells.

In a sixth aspect, the invention provides a method of delivering one or more cells cultured according the method of the fourth aspect, including the step of delivering the pharmaceutical composition of the fifth aspect to an individual to thereby facilitate renewal, cell migration and/or proliferation one or more cells in said individual.

It will be appreciated that in the aforementioned aspects that the one or more feeder-cell replacement factors is inclusive of biologically-active fragments thereof.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1 SDS-PAGE analysis of knock-out serum replacement (KSR) (Invitrogen) medium versus vitronectin:IGFBP3:IGF-1:bFGF (VN:GF-hES) medium 10% gradient polyacrylamide gel comparing VN:GF-hES medium versus KSR medium. Lanes contain: M) 250 kDa marker; 1) 0.1 μL KSR; 2) 10 μL VN:GF-hES medium. Molecular weight markers were sourced from Amersham Biosciences.

FIG. 2 Morphology of the hES cells and the MEF cells grown in KSR and VN:GF-hES culture conditions. The MEF cells were propagated using media containing A) KSR and E) VN:GF-hES. The hES cells were propagated using media containing B) KSR and F) VN:GF-hES. The hES cells express markers to mouse anti-Oct-4 antibodies when cultured in media containing C) KSR and G) VN:GF-hES. The hES cells express markers to mouse anti-Tra 1-81 antibodies when cultured in media containing D) KSR and H) VN:GF-hES (Scale bar=200 um).

FIG. 3 RT-PCR Analysis of mRNA isolated from hES cells grown in KSR and VN:GF-hES culture conditions (A) RT-PCR analysis of mRNA from hES cells grown in KSR culture conditions. Lanes contain: M) 100 by DNA ladder; 1) 18sRNA internal standard (151 by band); 2) 18sRNA negative control; 3) AP (177 by band); 4) AP negative control; 5) Oct-4 (169 by band); and 6) Oct-4 negative control. (B) RT-PCR analysis of mRNA from hES cells grown in VN:GF-hES culture conditions. Lanes contain: M) 100 by DNA ladder; 1) 18sRNA internal standard (151 by band); 2) 18sRNA negative control; 3) AP (177 by band); 4) AP negative control; 5) Oct-4 (169 by band); 6) and Oct-4 negative control.

FIG. 4 Two dimensional separation of the conditioned medium collected from the MEF cells alone. (A) The first dimension separation of the conditioned medium (CM) collected from the MEF cells. The first dimension separation involved injecting 0.8 mg of protein, concentrated from the MEF CM and separated using a 0-500 mM NaCl gradient. (B) Subsequent fractions were then collected and applied to a second dimension separation which involved a 0-100% ACN gradient as per material and methods section. The data shown is a representative of 3 replicate analyses performed.

FIG. 5 Two dimensional separation of the conditioned medium collected from the MEF:hES cell culture. (A) The first dimension separation of the CM collected from the MEF:hES cells. First dimension separation involved injecting 0.8 mg of protein, concentrated from the MEF:hES cell CM and separated using a 0-500 mM NaCl gradient. (B) Subsequent fractions were then collected and applied to a second dimension separation which involved a 0-100% ACN gradient as per materials and methods section. The data shown is a representative of 3 replicate analyses performed.

FIG. 6 Morphology and expression of cell surface markers on the passage 2 keratinocytes propagated using vitronectin:IGFBP3:IGF-I:EGF (VN:GF®-Kc) medium for proteomic analysis. Primary keratinocytes were isolated serum-free and then propagated using: (A) medium containing serum and a feeder cell layer, or (B) propagated serum-free using the VN:GF-Kc medium in conjunction with a feeder cell layer. Day 4 keratinocytes were probed with antibodies against: (C) keratin 6, and (D) keratin 14 to assess whether the primary keratinocytes propagated using the VN:GF-Kc remained undifferentiated. Conditioned media was collected from the cultures every two days from three different patient samples. (Scale bar=100 um) (n=3, images are of a representative culture of the 3 separate patients samples analysed).

FIG. 7 Two dimensional separation of conditioned media. Media was collected from (A) feeder cells alone and (B) feeder cell:keratinocyte cultures. First dimension separation involved injecting 1 mg of protein, concentrated from the conditioned media, onto a 0-500 mM NaCl gradient. Subsequently, fractions were collected and applied to a second dimension separation which involved using a 0-100% acetonitrile gradient as per the material and methods section. (conditioned medium from 3 separate patient cultures were pooled).

FIG. 8 Morphology and marker analysis of feeder and serum-free hES cells. hES cells were propagated for 15 passages and the differentiation of the cell was monitored via A) morphology, B) DAPI, C) SSEA-4, D) Oct4, E) SSEA1 and F) TRA1-60.

FIG. 9 Real time PCR analysis of transcripts expressed in undifferentiated stem cells. hES cells were propagated for 15 passages and real time PCR was conducted on Dppa, REX, TERT, UTF1, SOX2, FOXD4, Nanog and Oct4.

### DETAILED DESCRIPTION OF THE INVENTION

The present invention has evolved from a proteomic analysis of the paracrine interactions in a feeder cell-dependent system. More particularly, the inventors hypothesised that characterisation of the in vitro microenvironment of a feeder cell-dependent system would identify the factors produced by the feeder cells that are required for growth of the feeder-dependent cells. Vital to this proteomic approach is examination of the conditioned media using the VN:GF medium, which is fully defined and has minimal protein content. Use of such cell culture medium eliminates “masking” by exogenous protein of critical factors secreted by the feeder cells which may be important for supporting feeder-dependent cell growth.

Using this type of analysis, the inventors have identified several factors secreted by feeder cells in the aforementioned in vitro microenvironment. Hence, these factors can be used to formulate a well-defined non-cell-conditioned medium to culture cells, which obviates the need for feeder cells. Thus the present invention provides a significant advance in development of a feeder cell-independent cell culture system and medium for the growth of cells.

A person of skill in the art will appreciate that the invention is broadly applicable to any cell culture system for the growth of cells that is derived from human and non-human cells that can be grown in a feeder cell independent manner. By way of example only, the invention may be applied to murine ES cells.

In the context of the present invention, by “feeder cell replacement factor” is meant a protein which, when included in a cell culture medium, mimics, substitutes or replaces one or more functions and/or properties of a feeder cell. More particularly, the functions of interest include promoting attachment, propagation and/or maintenance of cell viability of a feeder-dependent cell, although without limitation thereto.

The invention further contemplates the use of biologically-active fragments of a feeder cell-replacement factor.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

In one embodiment, said “biologically-active fragment” has no less than 10%, preferably no less than 25%, more preferably no less than 50% and even more preferably no less than 75, 80, 85, 90 or 95% of a biological activity of a protein from which it is derived.

Due in part to the complex nature of paracrine interactions in a feeder cell-dependent system, there are vast array of proteins which are suitable for use as a feeder cell replacement factor as demonstrated by proteomic analysis of conditioned medium described herein. By way of example only, suitable feeder cell replacement factors include extracellular matrix proteins, growth factors, cell signalling and signal transduction proteins and growth factor receptors, although without limitation thereto.

In preferred embodiments, the one or more isolated feeder cell replacement factors are selected from the group consisting of human growth hormone, bone morphogenic protein 15, growth differentiation factor 9 (GDF-9), megakaryocyte colony-stimulating factor, secreted frizzled-related protein 2, Wnt-2b, Wnt-12, growth inhibitory factor, fetuin, human serum albumin (HSA), hepatocyte growth factor (HGF), transforming growth factor-α (TGF-α), TGF-β, nerve growth factor, platelet derived growth factor-β (PDGF-β), PC-derived growth factor (progranulin), interleukin (IL)-1, IL-2, IL-4, IL-6, IL-8, IL-10, IL-13 and Activin-A

More preferably, the one or more isolated feeder cell-replacement factors are selected from the group consisting of human growth hormone, bone morphogenic protein 15, growth differentiation factor 9, megakaryocyte colony-stimulating factor, secreted frizzled-related protein 2, Wnt-2b, Wnt-12, growth inhibitory factor and Activin-A.

Even more preferably, the one or more isolated feeder cell-replacement factor is Activin-A.

In other general embodiments, the one or more isolated feeder cell-replacement factor may be selected from the group consisting of the proteins listed in Table 1, Table 2, Table 3, Table 4 and Table 5.

Therefore, the present invention provides that one or more of the aforementioned feeder cell replacement factors are included in a cell culture medium for culturing a feeder-dependent cell. It is contemplated that formulation of the cell culture medium of the present invention relies upon use of one or more feeder cell replacement factors (as described herein) or other protein components that are isolated and/or synthetic.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

As used herein, by “synthetic” is meant not naturally occurring but made through human technical intervention. In the context of synthetic proteins, this encompasses molecules produced by recombinant or chemical synthetic and combinatorial techniques as are well understood in the art.

A particular advantage of this invention is that in preferred embodiments, the cell culture medium and system is amenable to addition of growth factors other than the one or more feeder cell-replacement factors.

Advantageously, such growth factors stimulate significant proliferative responses in primary cell cultures ex vivo in the absence of serum.

In general aspects, the cell culture medium of the present invention comprises a synthetic chimeric protein that stimulates cell migration and/or proliferation by binding and synergistically co-activating growth factor receptors (such as the IGF-I receptor) and VN-binding integrin receptors. In preferred embodiments, the synthetic chimeric protein comprises an IGF amino acid sequence and a VN amino acid sequence. Typically, although not limited thereto, the synthetic chimeric protein comprises a domain of mature VN that binds integrin receptors and an IGF, or at least a domain of IGF which can bind an IGF receptor. International Publication WO04/069871 provides non-limiting examples of suitable synthetic chimeric proteins and is incorporated herein by reference.

Preferably, the IGF amino acid sequence is an IGF-I amino acid sequence or an IGF-II amino acid sequence.

More preferably, the IGF amino acid sequence is an IGF-I amino acid sequence.

In preferred general embodiment, the VN amino acid sequence is any portion or domain of VN (and in particular mature VN) which is capable of binding an αᵥ integrin.

In preferred embodiments, the VN amino acid sequence is amino acid residues 1 to 64 of mature VN.

The present invention also contemplates inclusion of linker sequences in the aforementioned synthetic chimeric proteins (although without limitation thereto) as described generally in International Publication WO04/069871 provides general examples of suitable linker sequences and is incorporated herein by reference.

Preferably, said linker sequences comprises one or more glycine residues.

More preferably, said linker sequence further comprises one or more serine residues.

In a preferred embodiment, the linker sequence comprises Gly₄Ser.

In a particularly preferred embodiment, the linker sequence is (Gly₄ Ser)₄.

In a particularly preferred embodiment, the synthetic chimeric protein comprises IGF-I, a linker sequence of (Gly₄Ser)₄ and amino acid residues 1 to 64 of mature vitronectin (hereinafter referred to as IGF-I/1-64VN). In a particularly preferred embodiment, IGF-I/1-64VN is a single, contiguous protein.

In preferred embodiments, the cell culture medium of the present invention further comprises a growth factor in the form of an isolated IGF-containing protein complex wherein the IGF selected from the group consisting of IGF-I and IGF-II.

In another preferred embodiment that contemplates addition of an isolated IGF-containing protein complex where the IGF is IGF-II, the cell culture medium further comprises vitronectin.

In yet another preferred embodiment encompassing addition of IGF-I, the cell culture medium further comprises an IGFBP and VN.

Suitably, the IGFBP is selected from the group consisting of IGFBP-1, IGFBP-2, IGFBP-3, IGFBP-4, IGFBP-5 and IGFBP-6.

Preferably, the IGFBP is IGFBP-3 or IGFBP-5.

In embodiments where IGF-II and VN or IGF-I, IGFBP and VN are present, these proteins may be included as protein complexes, for example as described in International Publication WO02/24219.

It will be readily appreciated from the foregoing that isolated protein complexes of the invention may be in the form of non-covalently associated oligo-protein complexes or oligo-protein complexes that have been covalently cross-linked (reversibly or irreversibly), although not limited thereto.

Suitably, the one or more feeder cell-replacement factors are present in a concentration in the cell culture medium which facilitates cell growth and proliferation.

In general preferred embodiments, the or each isolated feeder cell-replacement factor is at a final concentration that is amenable to support cell viability, maintenance, renewal and/or proliferation and preferably between 0.1 ng/ml and 50 μg/ml. More preferably, the or each isolated feeder cell-replacement factor may be present at a final concentration of between 0.1 ng/ml and 50 μg/ml and more preferably at 1 ng/ml, 2 ng/ml, 5 ng/ml, 10 ng/ml, 15 ng/ml, 20 ng/ml, 25 ng/ml, 25 ng/ml, 30 ng/ml, 35 ng/ml, 40 ng/ml, 45 ng/ml, 50 ng/ml, 100 ng/ml, 150 ng/ml, 200 ng/ml, 250 ng/ml, 300 ng/ml, 350 ng/ml, 400 ng/ml 500 ng/ml, 600 ng/ml, 800 ng/ml, 1000 ng/ml, 1500 ng/ml and even more preferably 2 μg/ml, 3 μg/ml, 4 μg/ml, 5 μg/ml, 10 μg/ml, 15 μg/ml, 20 μg/ml, 25 μg/ml, 30 μg/ml, 35 μg/ml, 40 μg/ml 45 μg/ml and 50 μg/ml.

It will be readily appreciated that the invention is applicable to any cell type which is dependent on a feeder cell or other feeder cell-replacement techniques, for example, Matrigel or high extracellular matrix concentrations, for propagation.

Generally, such feeder-dependent cells are fastidious and require serum for growth and a supply of excretions and soluble factors from the feeder cells for growth and propagation. For example with reference to pluripotent cells or primary cell cultures, it may also desirable to maintain the cells in an undifferentiated state for further applications and in particular, therapeutic applications.

In one preferred embodiment, the feeder-dependent cell is a human embryonic stem cell.

In another preferred embodiment, the feeder dependent cell is a keratinocyte.

A particular advantage of the present invention is a feeder-independent cell culture system which does not require serum or requires very little serum.

Therefore in particular aspects, the invention provides a cell culture medium and system comprising one or more feeder-cell replacement factors, such that exogenous, animal-derived factors such as feeder cells and serum are not required or are required at substantially reduced levels, whereby cell growth and/or viability are maintained.

It will therefore be appreciated that “an absence of serum or an amount of serum which in the absence of said at least an IGF would not support cell growth” means either no serum or a substantially reduced amount or concentration of serum than would ordinarily be required for optimal cell growth and/or development in vitro.

By “serum” is meant a fraction derived from blood that comprises a broad spectrum of macromolecules, carrier proteins for lipoid substances and trace elements, cell attachment and spreading factors, low molecular weight nutrients, and hormones and growth factors. Operationally, serum may be defined as the proteinaceous, acellular fraction of blood remaining after removal of red blood cells, platelets and clotted components of blood plasma. The most widely used animal serum for cell culture is fetal bovine serum, FBS, although adult bovine serum, horse serum and protein fractions of same (e.g. Fraction V serum albumin) may also be used.

Typically, mammalian cells require between 5-10% serum depending on cell type, duration of culture, the presence or absence of feeder cells and/or other cellular components of a culture system and other factors that are apparent to persons of skill in the art.

Thus, in a preferred embodiment, the invention contemplates less than 5% serum, more preferably less than 2% serum, even more preferably less than 1% serum or advantageously no more than 0.5%, 0.4%, 0.3% or 0.2% serum (v/v).

In particularly advantageous embodiments, the invention contemplates no serum or no more than 0.5% or 0.25% serum (v/v).

Suitably, the culture medium of the invention may comprise other defined components. Non-limiting and in some cases optional components include well known basal media such as DMEM or Ham's media, antibiotics such as streptomycin or penicillin, human serum albumin (HSA), phospholipids (eg. phosphatidylcholine), sphingomyelin, activin-A, amino acid supplements such as L-glutamine, anti-oxidants such as β-mercaptoethanol, buffers such as carbonate buffers, HEPES and a source of carbon dioxide as typically provided by cell culture incubators.

The invention also contemplates use of additional biologically active proteins, or fragments thereof, that regulate cell growth, differentiation, survival and/or migration such as insulin-like growth factor-I (IGF-I), insulin-like growth factor-II (IGF-II), a laminin, epidermal growth factor (EGF; Heldin et al., 1981, Science 4 1122-1123), fibroblast growth factor (FGF; Nurcombe et al., 2000, J. Biol. Chem. 275 30009-30018), basic fibroblast growth factor (bFGF; Taraboletti et al., 1997, Cell Growth. Differ. 8 471-479), osteopontin (Nam et al., 2000, Endocrinol. 141 1100), thrombospondin-1 (Nam et al., 2000, supra), tenascin-C (Arai et al., 1996, J. Biol. Chem. 271 6099), PAI-1 (Nam et al., 1997, Endocrinol. 138 2972), plasminogen (Campbell et al., 1998, Am. J. Physiol. 275 E321), fibrinogen (Campbell et al., 1999, J. Biol. Chem 274 30215), fibrin (Campbell et al., 1999, supra) or transferrin (Weinzimer et al., 2001, J. Clin. Endocrinol. Metab. 86 1806).

Preferably, the invention provides a cell culture medium further comprises one or more additional biologically active proteins selected from the group consisting of EGF, bFGF, IGF-I, IGF-II and a laminin.

More preferably, the one or more additional biologically active proteins are selected from bFGF and a laminin.

It will be appreciated by the skilled addressee that laminins are a family of eukaryotic extracellular matrix glycoproteins which are composed of at least three non-identical chains (α, β, and γ chains) and a number of different isoforms resulting from various combinations of the α, β, and γ chains. Non-limiting examples of the different laminin isoforms include laminin-1, laminin-2, laminin-3, laminin-4, laminin-5, laminin-5B, laminin-6, laminin-7, laminin-8, laminin-9, laminin-10, laminin-12, laminin-13, laminin-14 and laminin-15, although without limitation thereto. It is also contemplated that in preferred embodiments, the laminin is a combination of laminin isoforms as hereinbefore described. It will be further appreciated that the laminin may be of any origin that is suitable for inclusion into a cell culture medium, particularly a cell-culture medium with potential therapeutic uses, such as mouse, pig, human, sheep but not limited thereto.

In particularly preferred embodiments, the laminin is as described in Catalogue No. CC095 from Millipore.

In preferred embodiments, the one or more additional biologically active proteins may be present at a final concentration of between 0.1 ng/ml and up to 50 μg/ml, 60 μg/ml, 70 μg/ml, 80 μg/ml, 90 μg/ml or 100 μg/ml. Preferably, the one or more additional biologically active proteins may be present at a final concentration of between 0.1 ng/ml and 50 μg/ml and more preferably at 50 ng/ml, 100 ng/ml, 200 ng/ml, 500 ng/ml, 1000 ng/ml, 1500 ng/ml and even more preferably 2 μg/ml, 5 μg/ml, 10 μg/ml, 15 μg/ml, 20 μg/ml, 25 μg/ml, 30 μg/ml, 35 μg/ml, 40 μg/ml and 45 μg/ml.

In particularly preferred embodiments which encompass laminin, the concentration of laminin is up to 50 μg/ml (or advantageously 25-100 μg per 10 cm² cell culture dish area) and preferably between 1 μg/ml and 40 μg/ml.

In general aspects, the invention provides an embryonic stem cell culture medium. In particular, the embryonic stem cell culture medium comprises between about 250 and 1000 ng/ml of a synthetic chimeric protein comprising an IGF amino acid sequence and a VN amino acid sequence, between about 50 and 100 ng/ml of bFGF, between about 25 and 50 ng/ml of Activin-A and between about 10 and about 50 μg/ml of a laminin.

In preferred embodiment, the cell culture medium of the present invention comprises about 1000 ng/ml of a synthetic chimeric protein an IGF amino acid sequence and a VN amino acid sequence, about 100 ng/ml of bFGF, about 35 ng/ml Activin-A and about 40 μg/ml laminin.

In a particularly preferred embodiment, the synthetic chimeric protein is IGF-I/1-64VN.

In light of the foregoing, a person of skill in the art will readily appreciate that any protein and in particular, the isolated feeder cell replacement factor, may be generated by way any suitable procedure known to those of skill in the art.

The invention further contemplates variants of the isolated feeder cell-replacement factors. In one embodiment, a “variant” has one or more amino acids that have been replaced by different amino acids. It is well understood in the art that some amino acids may be changed to others with broadly similar properties without changing the nature of the activity of the protein (conservative substitutions).

In one embodiment, a variant shares at least 50%, 60%, 70%, preferably at least 80%, more preferably at least 90% and advantageously at least 95%, 96%, 97%, 98% or 99% sequence identity with the amino acid sequences described herein.

Preferably, sequence identity is measured over at least 60%, more preferably at least 75%, even more preferably at least 90% and advantageously over substantially the full length of the synthetic protein of the invention.

In order to determine percent sequence identity, optimal alignment of amino acid and/or nucleotide sequences may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference.

In another example, “sequence identity” may be understood to mean the “match percentage” calculated by the DNASIS computer program (Version 2.5 for windows; available from Hitachi Software engineering Co., Ltd., South San Francisco, Calif., USA).

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The invention also contemplates derivatives of any protein described herein and in particular, of a feeder cell-replacement factor.

As used herein, “derivative” has been altered, for example by addition, conjugation or complexing with other chemical moieties or by post-translational modification techniques as are well understood in the art

“Additions” of amino acids may include fusion with other peptides or polypeptides. The other peptide or polypeptide may, by way of example, assist in the purification of the protein. For instance, these include a polyhistidine tag, maltose binding protein, green fluorescent protein (GFP), Protein A or glutathione S-transferase (GST).

Other derivatives contemplated by the invention include, but are not limited to, modification to side chains, incorporation of unnatural amino acids and/or their derivatives during protein synthesis and the use of crosslinkers and other methods which impose conformational constraints on proteins. Non-limiting examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

The imidazole ring of a histidine residue may be modified by N-carbethoxylation with diethylpyrocarbonate or by alkylation with iodoacetic acid derivatives.

Examples of incorporating non-natural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Further examples of chemical derivatization of proteins are provided in Chapter 15 of CURRENT PROTOCOLS IN PROTEIN SCIENCE Eds. Coligan et. al., John Wiley & Sons NY (1995-2001).

According to the invention, a protein may be prepared by any suitable procedure known to those of skill in the art.

It is contemplated that proteins of the invention may be in substantially pure native form.

In another embodiment, a protein may be produced by chemical synthesis. Chemical synthesis techniques are well known in the art, although the skilled person may refer to Chapter 18 of CURRENT PROTOCOLS IN PROTEIN SCIENCE Eds. Coligan et. al., John Wiley & Sons NY (1995-2001) for examples of suitable methodology.

In yet another embodiment, a protein may be prepared as a recombinant protein.

Production of recombinant proteins is well known in the art, the skilled person may refer to standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

Recombinant proteins may further comprise a fusion partner.

Well known examples of fusion partners include, but are not limited to, glutathione-S-transferase (GST), Fc portion of human IgG, maltose binding protein (MBP) and hexahistidine (HIS₆), which are particularly useful for isolation of the fusion protein by affinity chromatography. For the purposes of fusion protein purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In some cases, the fusion partners also have protease cleavage sites, such as for Factor Xₐ or Thrombin, which allow the relevant protease to partially digest the fusion protein of the invention and thereby liberate the recombinant protein therefrom. The liberated protein can then be isolated from the fusion partner by subsequent chromatographic separation.

Fusion partners according to the invention also include within their scope “epitope tags”, which are usually short peptide sequences for which a specific antibody is available. Well known examples of epitope tags for which specific monoclonal antibodies are readily available include c-myc, haemagglutinin and FLAG tags.

Suitable host cells for expression may be prokaryotic or eukaryotic, such as Escherichia coli (DH5α for example), yeast cells, Sf9 cells utilized with a baculovirus expression system, CHO cells, COS, CV-1, NIH 3T3 and HEK293 cells, although without limitation thereto.

Recombinant protein expression may be achieved by introduction of an expression construct into a feeder-dependent cell.

Typically, the expression construct comprises a nucleic acid to be expressed (encoding the recombinant protein) operably linked or operably connected to a promoter.

The promoter may be constitutive or inducible.

Constitutive or inducible promoters include, for example, tetracycline-repressible, ecdysone-inducible, alcohol-inducible and metallothionin-inducible promoters. Promoters may be either naturally occurring promoters (e.g. alpha crystallin promoter, ADH promoter, phosphoglycerate kinase (PGK), human elongation factor a promoter and viral promoters such as SV40, CMV, HTLV-derived promoters), or synthetic hybrid promoters that combine elements of more than one promoter (e.g. SR alpha promoter).

In a preferred embodiment, the expression vector comprises a selectable marker gene. Selectable markers are useful whether for the purposes of selection of transformed bacteria (such as bla, kanR and tetR) or transformed mammalian cells (such as hygromycin, G418 and puromycin).

Expression constructs may be introduced into feeder-dependent cells and in particular mammalian cells, by well known means such as electroporation, microparticle bombardment, virus-mediated gene transfer, calcium phosphate precipitation, DEAE-Dextran, cationic liposomes, lipofectin, lipofectamine and the like, although without limitation thereto.

For non-limiting examples of techniques potentially applicable to nucleic acid delivery to hES, reference may be made to Kobayashi et al., 2005, Birth Defects Research Part C: Embryo Today: Reviews, 75 10-18.

For non-limiting particular examples of methodology potentially applicable to expression of recombinant growth factor proteins in keratinocytes, reference may be made to Supp et al., 2000, J. Invest. Dermatol. 114 5 and Supp et al., 2000, Wound Repair Regen. 8 26-35.

### Pharmaceutical Compositions

The invention also provides pharmaceutical compositions that comprise one or more cells produced using the culture medium and/or system of the invention, such as hES cells and keratinocytes although not limited thereto, together with a pharmaceutically acceptable carrier diluent or excipient.

Pharmaceutical compositions of the invention may be used to promote or otherwise facilitate cell migration, tissue regeneration and wound healing.

Generally, the compositions of the invention may be used in therapeutic or prophylactic treatments as required. For example, pharmaceutical compositions comprising hES cells, keratinocytes or keratinocyte progenitor cells may be applied in the form of therapeutic or cosmetic preparations for skin repair, wound healing, healing of burns and other dermatological treatments.

Preferably, the pharmaceutically-acceptable carrier, diluent or excipient is suitable for administration to mammals, and preferably, to humans.

In particular embodiments, the pharmaceutical composition comprises autologous or allogeneic hES cells or keratinocytes cultured according to the invention.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion.

Controlled release formulations may be effected by coating, for example, with hydrophobic polymers including acrylic resins, waxes, higher aliphatic alcohols, polylactic and polyglycolic acids and certain cellulose derivatives such as hydroxypropylmethyl cellulose. Controlled release may be effected by using other polymer matrices, liposomes and/or microspheres. Non-limiting examples of controlled release formulations and delivery devices include osmotic pumps, polylactide-co-glycolide (PLG) polymer-based microspheres, hydrogel-based polymers, chemically-crosslinked dextran gels such as OctoDEX™ and dex-lactate-HEMA, for example.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

With regard to pharmaceutical compositions for wound healing, particular reference is made to U.S. Pat. No. 5,936,064 and International Publication WO99/62536 which are incorporated herein by reference.

In a particular embodiment relating to keratinocytes, the composition of the invention is suitable for spray delivery in situ.

The term “spray” encompasses and includes terms such as “aerosol” or “mist” or “condensate” that generally describe liquid suspensions in the form of droplets.

### Therapeutic Applications

One broad application of the cell culture medium, system and methods for propagation of feeder-dependent cells of the present invention includes therapeutic uses.

In particular aspects, the present invention contemplates methods for delivering one or more cells cultured produced according to aforementioned methods including the step of delivering the pharmaceutical compositions as herein before described to an individual.

The methods are particularly aimed at treatment of mammals, and more particularly, humans. However, it will also be appreciated that the invention may have veterinary applications for treating domestic animals, livestock and performance animals as would be well understood by the skilled person.

Therapeutic applications of hES cells cultured by the methods of the present invention include, but are not limited to, tissue regeneration, tissue transplantation or tissue renewal but are exclusive of methods that give rise to an entity that might reasonably claim the status of a human being. Non-limiting examples of such methods include methods for fertilising an ovum, methods for cloning at the 4-cell stage by division and methods for cloning by replacing nuclear DNA.

Non-limiting examples of therapeutic applications of ES, and in particular hES cells include Shufaro et al, 2004, Best Pract Res Clin Obstet Gynaecol 18(6):909-27.

In one preferred embodiment, the invention provides a culture medium, system and method for propagating primary keratinocytes ex vivo, which cells may be administered to an individual according to the invention.

In particular embodiments, the keratinocytes are autologous or allogeneic keratinocytes cultured according to the invention.

Such methods include administration of pharmaceutical compositions as hereinbefore defined, and may be by way of microneedle injection into specific tissue sites, such as described in U.S. Pat. No. 6,090,790, topical creams, lotions or sealant dressings applied to wounds, burns or ulcers, such as described in U.S. Pat. No. 6,054,122 or implants which release the composition such as described in International Publication W09/47070.

There also exist methods by which skin cells can be genetically modified for the purpose of creating skin substitutes, such as by genetically engineering desired growth factor expression (Supp et al., 2000, J. Invest. Dermatol. 114 5). An example of a review of this field is provided in Bevan et al., Biotechnol. Gent. Eng. Rev. 16 231.

Also contemplated is “seeding” a recipient with transfected or transformed cells, such as described in International Publication WO99/11789.

These methods can be used to stimulate cell migration and thereby facilitate or progress wound and burn healing, repair of skin lesions such as ulcers, tissue replacement and grafting such as by in vitro culturing of autologous skin, re-epithelialization of internal organs such as kidney and lung and repair of damaged nerve tissue.

Skin replacement therapy has become well known in the art, and may employ use of co-cultured epithelial/keratinocyte cell lines, for example as described in Kehe et al., 1999, Arch. Dermatol. Res. 291 600 or in vitro culture of primary (usually autologous) epidermal, dermal and/or keratinocyte cells. These techniques may also utilize engineered biomaterials and synthetic polymer “scaffolds”.

Examples of reviews of the field in general are provided in Terskikh & Vasiliev, 1999, Int. Rev. Cytol. 188 41 and Eaglestein & Falanga, 1998, Cutis 62 1.

More particularly, the production of replacement oral mucosa useful in craniofacial surgery is described in Izumi et al., 2000, J. Dent. Res. 79 798. Fetal keratinocytes and dermal fibroblasts can be expanded in vitro to produce skin for grafting to treat skin lesions, such as described in Fauza et al., J. Pediatr. Surg. 33 357, while skin substitutes from dermal and epidermal skin elements cultured in vitro on hyaluronic acid-derived biomaterials have been shown to be potentially useful in the treatment of burns (Zacchi et al., 1998, J. Biomed. Mater. Res. 40 187).

Polymer scaffolds are also contemplated for the purpose of facilitating replacement skin engineering, as for example described in Sheridan et al., 2000, J. Control Release 14 91 and Fauza et al., 1998, supra, as are microspheres as agents for the delivery of skin cells to wounds and burns (LaFrance & Armstrong, 1999, Tissue Eng. 5 153).

Keratinocyte sheets typically produced for therapeutic use are responsible for the ultimate closure of burn wounds. This sheet graft technique is applicable to all partial thickness burn injuries and is most useful in treating large surface area wounds where early permanent closure of both wound and donor sites is nearly impossible without external help. This is the type of injury responsible for the death of patients burnt in the recent Bali bombing.

Currently, it is possible to grow enough skin from a patient skin biopsy the size of a fifty-cent piece to cover an entire adult. This culture process takes 17 days. However, earlier skin replacement is urgently needed to reduce patient trauma, risk of infection, scarring and the present requirement for expensive temporary skin replacements ahead of permanent skin grafting. In addition, a sheet of cultured skin comprises many skin cells, some mature and some immature. The simple act of allowing cultured keratinocytes to reach confluence (necessary to produce sheets of skin) causes cells to prematurely loose their primitive characteristics i.e to differentiate. When a sheet of cultured skin is applied, only the immature cells are capable of attaching and establishing themselves on the patient. Because only small areas adhere, the sheets are very susceptible to damage arising from friction or movement of the patient and can sometimes result in the loss of the entire graft. Furthermore, in a sheet graft, the more mature skin cells in the sheet, the more likely it will be that the graft will not take and the cells themselves will not proliferate and migrate on the wound bed itself. Thus it is clear that earlier application of immature skin cells will result in better graft take and reduce scarring.

The present invention therefore provides a spray or aerosol delivery method to deliver skin cells cultured ex vivo onto a patient's burnt, ulcerated or wounded skin to enable a larger surface area of the patient's body to be covered by immature skin cells much earlier than existing sheet graft technology. This could be as early as only 7 days. This would also significantly reduce scar formation, shock and heat loss and would enable faster return of skin function in partial thickness and also full thickness burns.

Another treatment contemplated by the present invention is the treatment of burns patients to achieve early closure of full thickness wounds, because take of cultured skin on a wound that has removed both the surface (epidermal) and deep layer (dermis) of skin is poor. The invention contemplates use of dermal substitutes in conjunction with the spray-on-skin to effect early permanent closure of these most horrific injuries. Both biological and synthetic dermal substitutes are contemplated. For example, a de-epidermised, de-cellularised cadaveric-derived dermal scaffold comprising isolated protein complexes of the invention may be overlayed with a synthetic epidermis (dressing). After approximately 7 days the dermis the present inventors hypothesise that this dermis will be highly infiltrated by autologous endothelial cells. At this time, the synthetic dermis will be removed and the patient's own ex-vivo expanded fibroblasts and keratinocytes will be applied to the allo-dermis.

It is anticipated that the spray-on-skin, rather than epidermal sheets, will be successful as the dermal substitute will act as a nutritious stabilising scaffold promoting the migration and anchoring of skin cells and other important cells normally found in the skin. This will result in improved take of cultured skin cells in full thickness skin injuries

So that the invention may be readily understood and put into practical effect, the following non-limiting Examples are provided

### Examples

### Example 1

### Analysis of the Human Embryonic Stem Cell In-Vitro Micro-Environment

### Materials and Methods

### Mouse Embryonic Fibroblast Cell Culture

MEFs (SCRC-1046 cell line, Cryosite, Lane Cove, Sydney, NSW, AUS) were expanded to passage 6 on 80 cm² culture flasks (Nalge Nunc International, Rochester, N.Y., USA) using 85% Dulbecco's Modification of Eagle's Medium (DMEM) (Invitrogen, Mount Waverley, VIC, Australia) supplemented with 10% fetal bovine serum (FBS) (Invitrogen), 2×10⁻³ M L-Glutamine (Invitrogen) and 1000 IU/mL penicillin/streptomycin (Invitrogen) in 5% CO₂ at 37° C. Mitomycin-C (Sigma-Aldrich, Castle Hill, NSW, AUS) was subsequently added to the flasks containing the MEFs and the cells were incubated at 37° C. in 5% CO₂ for 2.5 to 3 hrs. Culture dishes (10 cm²) (Nalge Nunc International) were then coated in 0.1% gelatine (Sigma-Aldrich) for a minimum of 1 hr before the addition of the MEFs. MEF cells were seeded 20,000 cells/cm² onto 0.1% gelatin (Sigma-Aldrich)-coated 10 cm² (Nalge Nunc International) tissue culture dishes with 2.5 mL of MEF culture media per well.

The pre-attached MEF cells were serum-starved for two hours prior to changing to the serum-free media, VN:GF-hES. This medium consists of KO-DMEM (Invitrogen) containing 0.6 μg/mL VN (Promega, Annandale, NSW, AUS), 0.6 μg/mL IGFBP-3 (Tissue Therapies Ltd, Brisbane, QLD, AUS), 0.2 μg/mL IGF-I (GroPep, Adelaide, SA, AUS), 0.02 μg/mL basic fibroblast growth factor (bFGF) (Chemicon, Boronia, VIC, AUS), 2×10⁻³ M L-Glutamine (Invitrogen), 1000 IU/mL penicillin/streptomycin (Invitrogen), 1 μL/mL beta-mercaptoethanol (Sigma-Aldrich) and 12 ng/mL leukaemia inhibitory factor (LIF) (Chemicon). MEF cells were cultivated in a total of five 10 cm²/well culture dishes (Nalge Nunc-International) with 2.5 mL VN:GF-hES/well and incubated at 5% CO₂ at 37° C. The culture medium was changed daily, 48 hours post seeding the cells. After culturing the cells for 96 hours, approximately 150 ml of CM was collected.

### Human Embryonic Stem Cell Culture

The BG01V hES cells (ATTC, Manassa, Va., USA) were cultured on passage 6 mitomycin-C inactivated MEFs in hES cell medium containing KO-DMEM, 0.02 μg/uL bFGF (Chemicon), 2×10⁻³ M L-Glutamine, 1000 IU/mL penicillin/streptomycin, 1 μL/mL beta-mercaptoethanol, 12 ng/mL LIF and knock-out serum replacement (KSR) (Invitrogen). The hES cells were split 1:1 to 1:6 into 10 cm² culture dishes, depending on their rate of growth and confluence, using 0.05% trypsin/EDTA (Invitrogen) for 30 sec at 37° C. in 5% CO₂. Cells were then re-suspended in hES cell media and spun at 500-600 g for 5 min and transferred to a 10 cm² culture dish, pre-coated with 0.1% gelatin containing a passage 6 mitomycin-C inactivated MEF feeder layer. The hES cells and the feeder cells were re-fed every day from 48 hrs post transfer.

The serum-free culture of the hES cells involved the use of the previously mentioned inactivated MEF cells pre-plated in 10 cm² culture dishes (Nalge Nunc-International) and serum starved 2 hours before use. hES cells were then transferred to the serum starved MEFs in 2.5 mL of VN:GF-hES medium as described previously. Cultures were grown at 37° C. in 5% CO₂, and re-fed every day 48 hours after the initial transfer. Once cells were confluent, approximately 75 ml of CM was collected.

### Gel Analysis of KSR Versus VN:GF-hES Medium.

Protein content of the KSR versus VN:GF-hES-containing medium was compared using a 10% isocratic polyacrylamide gel. Briefly, samples were diluted to their appropriate concentrations, mixed in sample buffer (50 mL Glycerol/5 g SDS in 45 mL of TRIS-HCl/bromophenol blue) and were denatured at 100° C. for 10 mins. Lanes were loaded with 250 kDa Amersham markers (Amersham Biosciences, Piscataway, N.J., USA), 0.1 μL KSR medium (Invitrogen) and 10 μL VN:GF-hES medium. Proteins were separated using a 1× running buffer (25 mM Tris/200 mM glycine) at 100 Volts for 1 to 1.5 hrs. The gel was then silver stained for 30 min using the GelCode® SilverSNAP® stain Kit II (Pierce, Rockford, Ill., USA) until bands became visible and were then visualised using the G:BOX chemi (Syngene, Fredrick, Mass., USA).

### Immunofluoresence

Stage specific embryonic antigen-1 (SSEA-1), tumour repressor antigen 1-81 (Tra 1-81) and octamer-binding transcription factor-4 (Oct-4) are markers of pluripotency in hES cells [1, 2]. The presence of these markers was monitored to ensure that the CM was collected from undifferentiated hES cells. Cultures of hES cells were fixed using 2% paraformaldehyde/extraction buffer (0.5% triton X-100, 0.1 M Pipes buffer, 5 mM MgCl₂ and 1 mM EGTA at pH 7.0) for 10 min. The fixing agent was removed and the cultures were washed three times for 5 min in Dulbecco's phosphate buffered saline (PBS) (Sigma-Aldrich) to remove excess paraformaldehyde. Cultures were then incubated in 4% goat serum for 1 hr at 25° C. This solution was removed and primary antibodies to SSEA-1, Tra 1-81, and Oct-4 (Chemicon), diluted 1:50 in 4% goat serum and the cultures were incubated at 25° C. for 1 hr. The primary antibodies were removed and the washing steps were repeated. The anti-mouse secondary antibodies (Chemicon) were then diluted in PBS at 1:100 and were incubated for 1 hr. The secondary antibodies were removed, the wash steps were repeated and the colonies were photographed with a Nikon TE-2000 fluorescence microscope (Nikon, Lidcombe, NSW, AUS).

### RT-PCR Analysis

Reverse Transcriptase Polymerase Chain Reaction (RT-PCR) analysis was applied to detect transcripts of the Oct-4 and alkaline phosphatase (AP) genes to further analyse the differentiation status of the hES cells. RNA was isolated from the hES cell colonies using tri-reagent and its accompanying protocol (Sigma-Aldrich). RNA samples were then hybridised to oligo-dT 18 mers to create cDNA. The Oct-4 primers were: sense, 5′-CTTGCTGCAGAAGTGGGTG-GAGGAA-3′ (SEQ ID NO:1); and antisense, 5′-CTGCAGTGT-GGGTTTCGGG-CA-3′(SEQ ID NO:2). The alkaline phosphatase primers were: sense, 5′-TCAGAAGCTCAACACCAACG-3′(SEQ ID NO:3); and antisense, 5′-TTGTACGTCTTGGAG-AGGGC-3′(SEQ ID NO:4). The 18sRNA internal standard primers were: sense, 5′-TTCGGAACTGAGGCCATGA-T-3′ (SEQ ID NO:5); and antisense, 5′-CGAACCTCCGACTTTC-GTTCT-3′ (SEQ ID NO:6). One μg of cDNA was added to each of the four primer sets and subjected to an initial denaturation step of 94° C. for 5 min, followed by 35 cycles of denaturation at 94° C. for 30 sec, annealing at 55° C. for 30 sec and extension at 72° C. for 30 sec, followed by a final extension at 72° C. for 5 mins. Ten μL of the RT-PCR product were then analysed on 2% agarose gel at 100 Volts for 1 hr. Products were then visualised using ethidium bromide (Sigma-Adlrich).

### Two Dimensional Liquid Chromatography Proteomic Analysis.

Two-dimensional liquid chromatography was used to fractionate CM samples using the BioLogic Duo-flow system (Bio-rad, Hercules, Calif., USA) for first dimensional separation and the second stage of the Beckman Coulter's ProteomeLab™ PF 2D (Beckman Coulter, Gladesville, NSW, AUS) platform for second dimensional separation. Initially, the CM was acidified to pH 4 using 1.2 mL of 100% acetic acid and was concentrated using bulk-phase SPE phenyl-silica sorbant (Alltech-Australia, Dandenong South, VIC, AUS). Briefly, the matrix was prepared in 100% methanol and poured into a 10 cm³ gravity flow column (Bio-rad Laboratories) and equilibrated using 3 column volumes of ultra-pure water containing 0.1% acetic acid. Following this, samples were loaded onto the column (Bio-rad Laboratories) with proteins bounding to the resin via hydrophobic interactions. Bound protein was then eluted using 2 column volumes of 80% acetonitrile (ACN) in ultra-pure water containing 0.1% Trifluoroacetic acid (TFA) (Sigma-Aldrich). Eluted samples were then lyophilised using an eppendorf concentrator 5301 (Eppendorf South Pacific, North Ryde, NSW, AUS). The concentrated samples were reconstituted using 20 mM Tris-HCl and protein concentration was estimated using the Coomassie Plus Protein assay reagent (Pierce). Protein samples were then resolved in the first dimension using a UNO-Q (Bio-rad Laboratories) anion-exchange chromatography column attached to a BioLogic DuoFlow High Performance Liquid Chromatography system (Bio-rad Laboratories). Briefly, polypeptides were fractionated using a salt gradient (20 mM Tris-HCL through to 20 mM Tris-HCL containing 500 mM NaCl) and 1 mL fractions were collected at 2 min intervals using a flow rate of 0.5 mL/min.

Once the first dimension separation was complete, the anion-exchange fractions containing protein were further separated in the second dimension using high performance, reversed-phase liquid chromatography in a 30×4.2 mm non-porous silica C18 column. Reversed phase chromatography was performed by injecting 200 μL samples from each fraction onto the ‘ProteomeLab™ PF 2D (Beckman Coulter). Injected samples were fractionated independently using a 0-100% ACN/0.1% TFA gradient over 30 mins, collecting one minute fractions between 4 and 24 mins. Flow rates and column temperature were maintained at 0.75 mL/min and 50° C., respectively, for all separations. Two-dimensional images were generated for both the MEF CM and the MEF:hES cells CM samples using ProteoVue software (Eprogen, Darien, Ill., USA).

### Sample Preparation and MALDI-TOF-TOF Mass Spectrometry.

Once the samples were fractioned in the 2D chromatography workflow, 400 μL of each fraction was collected from their respective second dimension 96 well plates, and lyophilised as previously described. Lyophilised samples were then reduced, alkylated and tryptic-digested. Reduction was performed by resuspending the lyophilised protein in 100 μL of reduction buffer (0.1 M NH₄CO₃/20 mM DTT pH 7.9) and then incubating the samples at 56° C. for 1 hr. Alkalylation was performed by adding 10 μL of 50 mM iodoacetamide (Sigma-Aldrich) to the reduced sample and incubating the sample in the dark at 37° C. for 30 mins. The proteins were then digested using 2.2 μL of sequencing grade modified trypsin (Promega) and incubated in the dark at 37° C. overnight. The samples were then desalted using micro C18 ZipTips (Millipore, Bedford, Mass., USA) and the peptides were eluted directly with 5 mg/ml of alpha-cyano-4-hydroxy cinnamic acid (CHCA) in 60% ACN/0.1% TFA onto a Matrix-Associated Laser Desorption Ionization (MALDI) plate. A 10-fold dilution of the standard calibration mix was used as the calibrant for the MALDI plates on which the tryptic digest samples were spotted. The sample matrix used was CHCA at a concentration of 5 mg/ml in 50% acetonitrile in 5 mM ammonium phosphate and 0.1% TFA (Sigma-Aldrich). Samples were then analysed using a 4700 Proteomics Analyser MALDI-TOF-TOF (Applied Biosystems, Foster City, Calif., USA) at the Institute for Molecular Bioscience (St Lucia, QLD, Australia). All mass spectrometry (MS) spectra were recorded in positive reflector mode at a laser energy of 3200 μJ/pulse. For MS data, 1000 shots were accumulated for each spectrum obtained from the 4700 TOF-TOF MS/MS. All MS data from the TOF-TOF was acquired using the default 1 kV MS/MS method at a laser energy of 4500 μJ/pulse. The information obtained from non-interpreted TOF-MS and TOF-TOF MS/MS data was used to query mammalian entries in the MSDB database and was performed with the GPS Explorer™ (Applied Biosystems) automated interrogation of the MASCOT database. When searching peptide masses, the following parameters were set: missed cleavages=2; peptide tolerance=+/−0.5; enzyme=trypsin; variable modifications include oxidation of methionines; fixed modifications include carbamindomethyl of cysteine. The maximum number of hits were chosen and the proteins were analysed using protein score, protein score confidence interval, total ion score (TIS) and total ion score confidence interval.

Briefly, proteins were firstly ranked by TIS. This score indicates how well the proteins are matched on a sequence data base obtained from MS/MS analysis, with scores≧38 considered significant (p<0.05 that protein sequence data was matched randomly). Protein matches with scores≧38 where included for further analysis, however, when MS/MS data was not obtained, proteins were ranked based on protein score. This score indicates how well peptide masses match predicted trypsin cleaved peptide sequences, with scores≧60 considered significant (p<0.05 that masses were matched randomly). Proteins were selected based on the highest protein score, however, numerous protein scores≦60 were reported. These fractions were still included for further analysis. Furthermore, the reported function of each protein was examined using Swiss-Prot, PubMed, and Online Medelian Inheritance in Man (OMIM) searches.

Sample Preparation and LC/MS using LC/ESI/MS and LC-MALDI Analysis

Initially, first dimension fractions and raw samples (concentrated conditioned medium) were lyophilized using an eppendorf concentrator 5301 (Eppendorf South Pacific) for LC/ESI/MS and LC/MS, respectively. Lyophilised samples were then reduced, alkylated and digested with trypsin as previously described. The samples for Liquid Chromatography (LC) were then dissolved in 50/50 solvent A/B (solvent A 0.1% Formic acid) (solvent B 90% acetonitrile in 0.1% Formic acid). Samples were loaded onto a C18 300A column (150 mm×0.5 mm×5 μm particle size) (Vydac, Hesperia, Calif., USA) with 40/60 solvent A/B at a flow rate of 300 μL/min. Solvent delivery was achieved by using an Agilent 1100 Binary HPLC system (Agilent, Inc Santa Clara, Calif., USA).

Electrospray mass spectrometry was performed using a 4000 ESI-QqLIT mass spectrometer (Applied Biosystems) equipped with an atmospheric ionisation source (Applied Biosystems) at the Institute of Molecular Biosciences. Data was acquired using the Analyst 1.4.1 software (Applied Biosystems). The protein analysis was conducted using the MASCOT database GPS Explorer™ software (version 4.0) as previously described, with the mass/ion peak information obtained from both the MS and the MS/MS spectra. Briefly, the score is −10*Log(P), where P is the probability that the observed match is a random event. Individual ions scores>38 indicate identity or extensive homology (p<0.05).

Alternatively, samples collected from the LC phase were spotted onto MS plates using 1:1 volume of 5 mg/mL of CHCA (Sigma-Aldrich):protein sample for LC-MALDI analysis. Plates were analysed using the 4700 Proteomics Analyser (Applied Biosystems) at the Institute for Molecular Bioscience. A plate-wide calibration for MS and MS/MS data was performed using mass standards contained in the MS/MS Mass Standards kit (Sigma-Aldrich). Potential protein matches were then identified from automated searching of the MASCOT database using GPS Explorer™ protein analysis software (version 4.0) as previously described, with the mass/ion peak information obtained from both the MS and the MS/MS spectra. The function of each protein was then examined as previously described.

### Results

### Protein Content in VN:GF-hES Medium Versus KSR-Containing Medium.

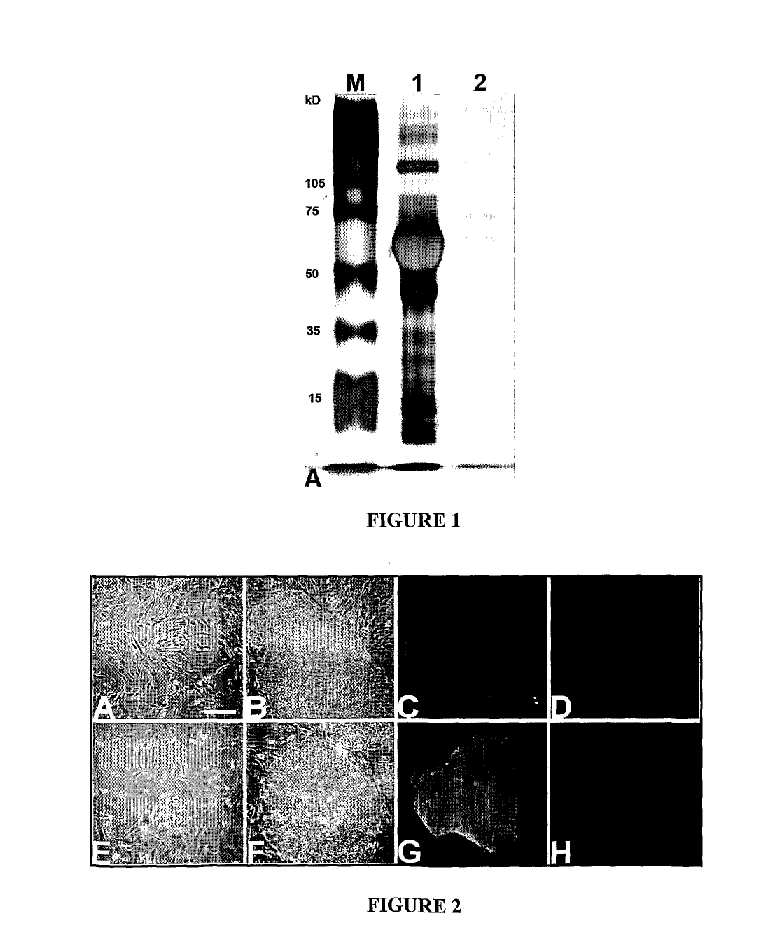

hES cells were initially resuscitated from frozen storage and then cultured on MEF cells using 20% KSR-containing medium. However, it was recognised that high abundant proteins in serum, such as serum albumin may effect a planned proteomic analysis by masking critical factors. Therefore, a serum-free medium, VN:GF-hES was employed, for the culture of the cells. In order to compare the total protein content of KSR versus VN:GF-hES, PAGE analysis using a 10% isocratic polyacrylamide gel was performed. This analysis revealed that VN:GF-hES (FIG. 1 lane 2) contains minimal protein compared to KSR (FIG. 1 lane 1) which clearly contains numerous unidentified proteins.

### Morphology of the hES Cells and the MEF Cells Grown in VN:GF-hES Media.

It has previously been demonstrated that hES cells can attach, expand and survive in an undifferentiated state when using VN:GF-hES medium as a serum-free media (Richards 2003 unpublished data). To ensure that the conditioned media (CM) to be analysed was collected from undifferentiated hES cells, morphological examination of the cells was performed. This experiment revealed that the VN:GF-hES propagated hES cells maintained tight compacted colonies that resembled those grown in KSR containing medium (FIGS. 2F and 2B, respectively). Furthermore, MEF cells propagated in the serum-free medium demonstrated similar morphology to those propagated in KSR. (FIGS. 2E and 2A, respectively).

### Marker Expression of the hES Cells Grown in VN:GF-hES Media.

At present there are no definitive markers to characterise the pluripotency of hES cells. However, hES cells express several markers, such as SSEA-4, Oct-4 and TRA1-81, all of which are unique to undifferentiated hES cells. These markers, taken together, are routinely used to verify that hES cells are phenotypically undifferentiated [29]. To confirm that the cells grown in the VN:GF-hES medium were undifferentiated, antibodies to TRA 1-81 and Oct-4, were selected to analyse the differentiation status of the hES cells. Both Oct-4 (Green) and TRA 1-81 (Red) revealed high levels of protein expression on the hES cells cultured under KSR conditions and VN:GF-hES conditions (FIGS. 2C and 2G and FIGS. 2D and 2H, respectively). Additionally, fluorescently labelled, secondary antibodies to SSEA-4 (expressed by undifferentiated cells), as well as fluorescently labelled, secondary antibodies to SSEA-1 (not expressed by undifferentiated cells), were selected to further analyse hES cell differentiation status. This analysis revealed that SSEA-4 was expressed on the cells cultured in either KSR or in the VN:GF-hES medium (data not shown). Similarly, SSEA-1 was not expressed on cells cultivated in either KSR or in the VN:GF-hES medium (data not shown). No immunoreactivity was observed when the hES cells were incubated with the secondary antibody alone (data not shown).

### RT-PCR Analysis of the hES Cells Grown in VN:GF-hES.

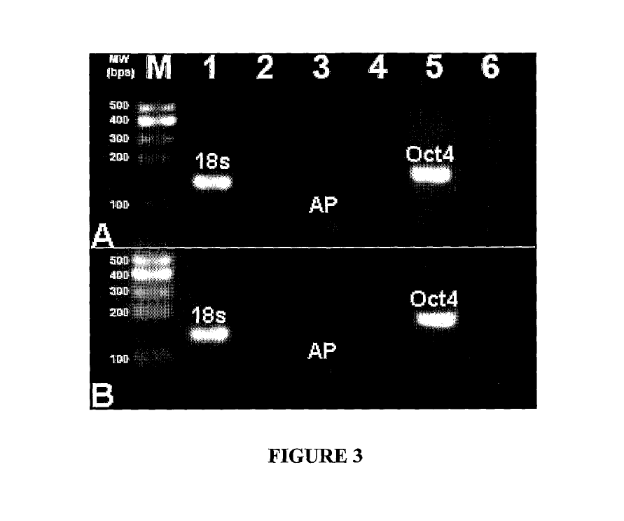

The expression of TRA 1-81, Oct-4 and SSEA-4 together is not definitive for identifying an undifferentiated hES cell colony. Therefore, RT-PCR analysis on the expression of two genes, Oct-4 and AP (alkaline phosphatase), was performed to further verify the differentiation status of the hES cells. In order to establish that the samples were not contaminated with complementary deoxyribose nucleic acid (cDNA) or genomic deoxyribose nucleic acid (gDNA), the template was omitted in the series of negative controls (data not shown). The primers were designed such that they annealed to different exons within the gene, so that any contaminating genomic deoxyribose nucleic acid (gDNA) present in the PCR reaction would result in a larger molecular weight band than the cDNA. This analysis revealed that the hES cell colonies grown in the KSR (FIG. 3A) and VN:GF-hES medium (FIG. 3B) expressed mRNA for Oct-4, AP and 18sRNA (included as an internal standard control). This experiment provides further verification that the CM collected for proteomic analysis was collected from undifferentiated hES cells.

Two Dimensional Separation of the Conditioned Media Collected from Both the MEF Alone and the MEF:hES Cell Cultures.

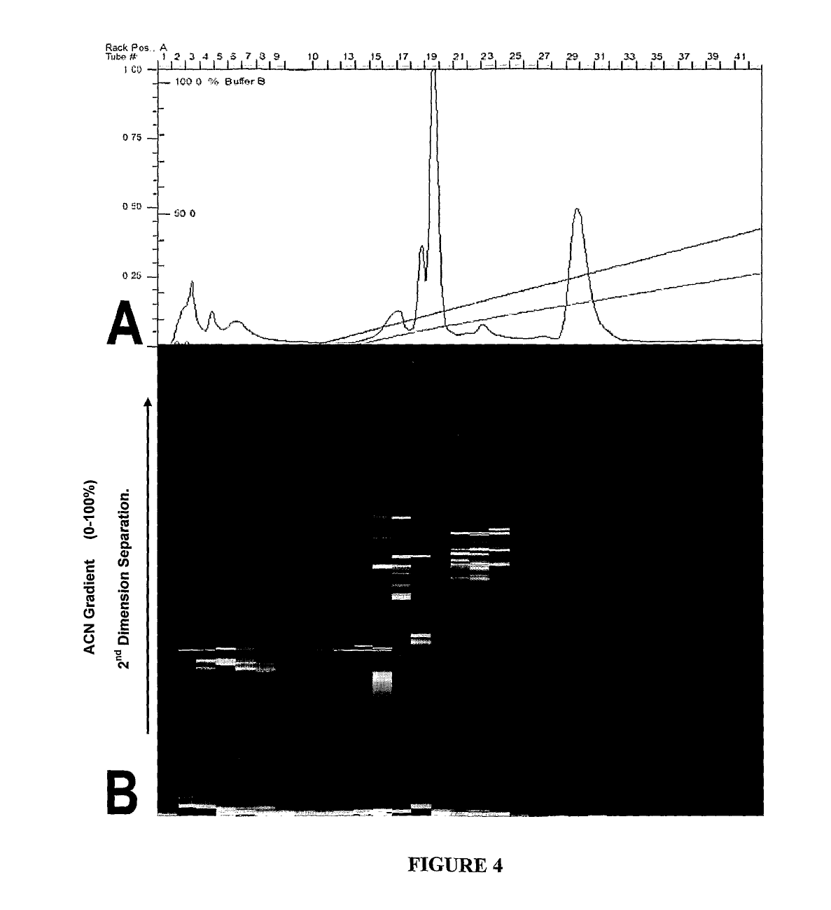

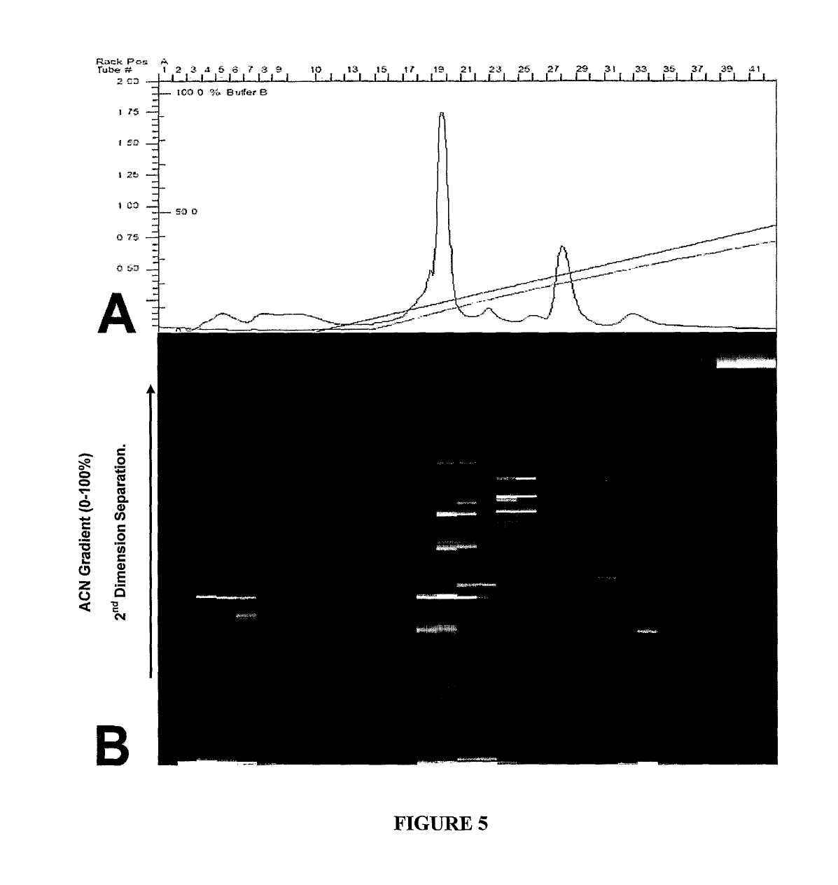

Proteins present in the CM from the MEF cells alone (FIGS. 4A and 4B) and the MEF:hES cells (FIGS. 5A and 5B) were separated using a novel form of two dimension liquid chromatographic separation. This process involved separating proteins via a salt gradient in the first dimension. First dimension fractions containing protein were then analysed using a second dimension separation approach employing a H₂0 ACN gradient. Proteins were then visualised using the ProteomeLab™ software package ProteoVue. Clear differences in protein profiles were evident between the MEF cells alone CM (FIG. 4B) and the MEF:hES cell CM (FIG. 5B). Several proteins were observed in the CM through this proteomic approach, however, only proteins which may be relevant to the hES cell in-vitro micro-environment are discussed herein. A total of 192 proteins from the MEF cells alone and 247 proteins from MEF:hES cells were identified from 3 separate two dimension chromatographic profiles (FIGS. 4B and 5B, respectively). These proteins were then isolated, digested and subjected to MALDI-TOF-TOF analysis. In addition, 35 fractions from MEF CM and 38 fractions from MEF:hES cell CM were isolated from their first dimension separation, digested and transferred to LC/ESI/MS (FIGS. 4A and 5A, respectively). Furthermore, 1 raw sample (concentrated, lyophilised and tryptic-digested), from MEF CM and 1 raw sample from MEF:hES cell CM were processed and subjected to LC-MALDI.

Identified Proteins from the MEF Cells Alone and the MEF:hES Cell Conditioned Media.

Proteins in the MEF CM and the MEF:hES cell CM were analysed using three methods, MALDI-TOF-TOF, LC/ESI/MS and LC-MALDI. The Mascot database was employed to analyse proteins present within the CM and the results were organised into seven protein species; ECM, membrane, nuclear, secreted, differentiation and growth factors, and serum-derived. Additionally, the proteins were categorised using accession number, molecular weight, protein score and ion score. The MALDI-TOF-TOF results were related to the protein score. The MALDI-TOF-TOF results for the MEF CM media revealed 3 ECM, 3 membrane, 3 nuclear, 1 cytoplasmic, 4 secreted and 7 differentiation and growth factor proteins (Table 1). Furthermore, the MALDI-TOF-TOF results for the MEF:hES cells CM revealed 4 membrane, 4 nuclear, and 6 secreted proteins (Table 2). All MALDI-TOF-TOF results, except for the nuclear protein heterogeneous nuclear ribonucleoprotein M (Table 2), were unconfirmed as determined by their protein scores. The LC/ESI/MS results are related to the ion scores. The LC/ESI/MS results for the MEF CM revealed 11 ECM, 4 membrane, 5 nuclear, 1 cytoplasmic, 3 secreted, and 3 serum-derived proteins (Table 1). Additionally, the LC/ESI/MS results for the MEF:hES cell CM revealed 12 ECM, 4 membrane, 6 nuclear, 6 cytoplasmic, 1 secreted, and 2 serum-derived proteins (Table 2). The LC-MALDI results are also related to the ion score. The LC-MALDI results for the MEF CM revealed 1 ECM, 1 membrane, 2 cytoplasmic and 1 secreted protein (Table 1). Furthermore, the LC-MALDI results for the MEF:hES cell CM revealed 1 cytoplasmic and 2 secreted proteins (Table 2). All proteins revealed via the LC/ESI/MS analysis were either confirmed or exhibit extensive homology as determined by their ion scores. All three analyses described above were conducted in order to increase the legitimacy of the returned results.

### Discussion

Since their first derivation in 1998 [1], hES cells have become one of the most promising sources of in-vitro cells for tissue replacement and repair. However, these “primitive” cells require xeno-derived components, such as MEF cells and bovie serum, to maintain undifferentiated propagation. This poses significant problems as patients receiving products derived from these cells may inadvertently be infected with diseases such as “new variant Creutzfeldt-Jakob disease”, which may be present in these poorly-defined and/or xeno-derived culture components. Moreover, Dr. Ajit Varki demonstrated that hES cell lines propagated in these xenogeneic culture conditions acquired a non-human sialic acid Neu5Gc, which was thought to have come from the MEF feeder cells [30]. This finding unequivocally demonstrates that hES cells are vulnerable to factors present in their in-vitro micro-environment. Therefore, if the therapeutic potential of these cells is to be met, these xeno-derived components clearly need to be eliminated from the current culture systems.

In light of this, many investigators worldwide have attempted to develop culture systems that are fully defined and xenogeneic-free. Recently Ludwig et al. (2006) discovered a method, termed TeSR1, for the feeder-free derivation and propagation of hES cell lines [17]. Whilst the TeSR1 method proved successful for the in-vitro propagation of hES cells, substantial quantities of proteins were needed, such as 13 mg/ml HSA and 23 μg/mL of insulin. This is far from ideal since high concentrations of growth factors may induce the hES cells to become tumourigenic. Furthermore, given that albumin is a carrier protein, it is very likely that at high concentrations purified HSA may carry other, as yet unidentified proteins. Therefore, this TeSR1 technology will not be commercially viable for the scaled up propagation of hES cells. Additionally, Ludwig et al. (2006) observed a 47XXY karyotype when the hES cells were cultured in the TeSR1 culture system for 5 months, thus rendering these cells therapeutically unviable [17]. A fully defined serum-free medium, termed VN:GF-hES, for the serial propagation of hES cells enables the long term propagation of hES cells in an undifferentiated state [31]. However, the self-renewal of the hES cells still requires the use of MEFs, thus highlighting the importance of these cells to the hES cell in-vitro micro-environment.

To date, it is still not established what role the MEF cells provide in the hES in-vitro micro-environment. However, it has been demonstrated that i3T3, a mouse embryonic fibroblast cell line used for the expansion of skin keratinocytes, secrete large quantities of IGFs and ECM proteins [32], as well as a variety of other components. Therefore, it can be assumed that the MEFs are secreting similar proteins important for the self-renewal of hES cells. Increasingly, a wide variety of these ECM proteins have been evaluated to promote a self-renewing environment for the hES cells. These proteins include purified collagens [33], laminins [18, 33], fibronectin [20, 33] and Matrigel™ [33]. However, these ECM culture technologies have only proved successful with the addition of other growth promoting components to the medium. Highlighting this, Xu et al. (2001) discovered that laminin and Matrigel™ only proved successful for the propagation of hES cells when used in conjunction with media conditioned by MEFs [18]. This suggests that the growth promoting components important for the survival of hES cells maybe secreted by the MEFs into the CM.

In view of this, the inventors hypothesised that the MEF feeder cells secrete novel proteins important for the self-renewal of hES cells and that these proteins may be identified through the use of advanced proteomic technologies. Recently, Prowse et al. (2005) and Wee Eng Lim and Bodnar (2002) analysed the proteomic profiles of fibroblast feeder cells and their respective CM. These studies have provided some preliminary insights into what the fibroblast cells may provide to the hES cells in-vitro micro-environment. However, this study takes this one step further by analysing not only the CM from the MEFs, but also the CM from the co-culture of MEFs with hES cells.

As reported herein, it was re-validated that the fully defined media, VN:GF-hES, supported the undifferentiated growth of the hES cells (FIG. 1-3). The present study demonstrated that the culture of cells in VN:GF-hES, rather than in KSR-containing media, led to an improved resolution of proteins within the CM (FIG. 1). In addition, this analysis clearly demonstrates that the VN:GF-hES medium had minimal protein content. In contrast, SDS-PAGE analysis revealed the presence of many high abundant proteins within the KSR-medium; some of which may have potentially masked critical factors secreted by the cells (FIG. 1 lane 1). Nevertheless, despite the minimal protein content of the VN:GF-hES medium, subsequent mass spectrometry analysis revealed that several bovine serum proteins, such as alpha-2-HS-glycoprotein and albumin, were still present within the CM collected from cells cultured serum-free in the VN:GF-hES medium. This was not entirely unexpected as the cells were cultivated in bovine serum containing medium prior to transferring cells to the VN:GF-hES medium. Furthermore, high abundant serum proteins, such as albumin are often adhesive and associate with extracellular surfaces and culture vessels, thus making them difficult to completely remove through washing steps alone. Moreover, the proteomics analyses reported by Prowse et al. (2005) and Lim and Bondar (2002) also observed several bovine serum proteins, even though they too adopted a series of washes prior to the incubation of cells in the serum-free medium. Nevertheless, it is clear that the series of washes and serum-starvation steps employed in both their study and the present study, prior to transfer to serum-free conditions significantly reduced the presence of these serum-derived components in the CM. Therefore, these bovine proteins did not markedly interfere with the resolution of the proteomic profiles (FIG. 3B and FIG. 4B). Nevertheless, there is an important distinction in the present approach compared to other proteomic analyses that should be highlighted herein; namely, the CM collected in the present study employed a medium that was optimal for cell growth i.e. contained VN:GF-hES. In contrast, the proteomic strategy used by Prowse et al. (2005) and Lim and Bondar (2002) employed basal, serum-free medium without mitogenic supplements i.e. the CM in previous studies was collected from cells that were “starved” and/or “stressed” and hence were not in optimal growth conditions.

The analysis of the CM reported herein, also positively identified a number of proteins within CM not normally associated with cell secretions, such as extracellular matrix proteins (ECM) from both the MEF cells alone and the MEF:hES cell cultures. The presence of these proteins may be due to proteolytic events. For example, collagenase 3 activity was tentatively identified within the MEF:hES cell CM. In addition, several of the ECM proteins confirmed in this study are commonly used in feeder-free culturing systems and support the attachment and proliferation of hES cells. These include collagen I and IV, fibronectin 1 [20], laminin M, laminin alpha 1, 4 and 5 [21, 18, 34] and proteoglycan [18] (Table 1 and 2). Furthermore, several of the above ECM proteins are analogous to the ECM proteins positively identified by Prowse et al. (2005) and Lim and Bondar (2002) in human and animal feeder cell CM, thus reinforcing the potential importance of these proteins in the CM. In addition, thrombospondin 1, confirmed in the MEF CM and also positively identified by Prowse et al. (2005) in human feeder cell CM, has demonstrated roles in cellular adhesion, migration and proliferation [35, 36]. The thrombospondin 1 gene is known to act synergistically with platelet derived growth factor (PDGF) [37], bFGF [37] and transforming growth factor-beta (TGF-beta) [38], three growth factors with significant roles in the self-renewal of hES cells. Other ECM proteins of interest include collagen V, VII, XI, XII and XV, tenascin X, and versican core protein. Whilst these proteins have not been investigated in hES cell culture, they each have critical functions, such as cellular attachment, proliferation and migration and thus may also contribute to an environment supportive for hES cell self renewal.

As previously discussed, ECM proteins have only proved successful in supporting hES cell expansion when supplemented with growth promoting agents. Importantly, the MEF cell CM revealed several growth factors relevant to the self-renewal of hES cells, such as IGF-I, IGF-II, TGF-beta 2, PDGF, bone morphogenetic protein 15 (BMP15), epidermal growth factor (EGF) and hepatocyte growth factor (HGF) (Table 1). To date, one of the most prominent growth components added to the hES cell culture is insulin [39]. Interestingly, it has been demonstrated that IGF-I is able to replace the need for insulin during the culture of keratinocytes [26]. Indeed, IGF-I is a major component of the VN:GF-hES serum-free medium and has been demonstrated to replace the need for insulin in the hES cell culture medium [31]. Furthermore, previous studies has demonstrated that a synergistic interaction between IGF-II and vitronectin (VN) results in increased cellular migration [40, 41]. Preliminary studies suggest that IGF-II may also promote the proliferation and self renewal of hES cells [31]. TGF-beta and PDGF have also been demonstrated to have roles in maintaining hES cells in an undifferentiated state [16, 23]. Interestingly, Hollier et al. (2005) and Schoppet et al. (2002) demonstrated that both these heparin-binding growth factors have the ability to bind and interact with VN [26, 42]. Other growth factors observed in the CM through the present proteomic analysis include EGF and HGF. These have also been reported to activate differentiation in hES cells [43]. However, bFGF, a common self renewal component added to hES cell culture [44, 45], has been also reported by Schuldiner et al. (2000) to induce hES cell differentiation. This data suggests that growth factors may have opposing effects on differentiation, depending on their concentrations and/or levels of expression. Thus, EGF and HGF, two growth factors shown to invoke differentiation, may also promote a self renewing environment for hES cells when used at appropriate concentrations. With respect to the BMP15 observed in the CM, there are no current studies which have investigated the relationship between this growth factor and hES cells. However, BMP4 has been demonstrated to promote hES cell differentiation [46]. Therefore, if BMP15 has similar effects to BMP4, the addition of antagonists, such as noggin [47], follistatin [48], Activin A [49] and bFGF [22], could be added to the culture medium to provide a self-renewing micro-environment for hES cells. Thus, it is clear that many of the proteins observed through this proteomic approach may be candidate factors that could be used in conjunction with the VN:GF-hES medium to remove the need for hES cells to be co-cultured with MEF cells.

If these growth factors are to prove useful for the propagation of hES cells, their respective receptors must also be expressed. The proteomic analysis of the CM reported herein, revealed several growth factor receptors, including fibroblast growth factor receptor (FGFR) and the insulin receptor (IR), both of which are relevant to hES cell growth [50, 39]. However, this study also revealed growth factor receptor-bound protein 14 (Grb 14) which has been demonstrated to inhibit the activities of the FGF [51, 52] and insulin [53-55] receptors. Therefore, the expression of Grb14 in hES cells may modulate the deactivation of critical signalling pathways triggered by the FGF and insulin receptors and indirectly the self-renewal of hES cells.

Whilst individual proteins can be identified to support the growth of cells in a feeder-free system, it is clear that many of these candidates are involved in complex pathways and signalling events. For example, Wnt2b and secreted frizzled-related protein 2, found in the MEF CM is known to have roles in Wnt/13-catenin signalling, which is important for hES cell self renewal [56]. Wnt2b appears to elicit this function by stabilising B-catenin, thereby activating transcription of Tcf/LEF target genes [57] and secreted frizzled-related protein 2 by inhibiting secreted frizzled-related protein 1 which limits the Wnt signalling pathway [58]. Furthermore, casein kinase I (isoform alpha), found in the MEF:hES cell CM, has been demonstrated to improve stabilisation of beta-catenin and the induction of genes which are targets of Wnt signals [59]. Casein kinase I (isoform alpha) has also been reported to bind and increase the phosphorylation of dishevelled [60], a known component of the Wnt pathway and present in the MEF:hES cell CM. TBX20, a transcription factor, identified in the MEF CM, has also been demonstrated to positively regulate the Wnt pathway [61]. Clearly, complex interactions occur within this pathway; however future studies and analyses may reveal a component/s in the activation of Wnt/B-catenin signalling that may drive hES cells to self-renew.

Additionally, tumour rejection antigenl (Tra1) homolog and follistatin-related protein 1, both positively identified in the CM (Table 1, 2) have roles in the regulation of human Telomerase Reverse Transcriptase (hTERT), thus has been linked to the self-renewal of hES cells [62]. Tra1 homolog and the myc-binding protein 2, also positively identified in the MEF:hES cell CM, has been reported to activate hTERT through the regulation of c-myc [63]. Interestingly, follistatin-related protein 1, has been demonstrated to inactivate activin-A [64] and TGF-β, two proteins which have been demonstrated to suppress hTERT [65]. Conversely, the activin/TGF-β/nodal branch has been demonstrated to induce hES cell self-renewal [16]. Hence, the above studies suggest that while TGF-β inhibits hTERT, therefore inducing differentiation, it also acts in conjunction with activin to promote pluripotency in hES cells, thus highlighting the complexities that exist within regulatory pathways.

Another pathway important to the self-renewal of embryonic stem (ES) cells is the signal transducer and activation of the transcription 3 (STAT3) pathway [66]. This study has revealed two proteins in the CM that are known to be involved in the regulation of STAT3; namely E3 SUMO (inhibits) and EGF (activates). E3 Sumo appears to inhibit the regulation of the STAT3 pathway by blocking DNA-binding activity of STAT3 [67], whereas EGF appears to induce the tyrosine phosphorylation and nuclear translocation of STAT3 in mouse liver cells [68]. Several other studies have revealed that the addition of cytokines, such as interleukin (IL)-6, can activate the gp130 receptor and induce phosphorylation of STAT3 in mouse ES cells [69]. However, IL-6 has failed to elicit the same responses in hES cells [70]. Nevertheless, several cytokines, such as IL-1, IL-2, IL-4, IL-8, IL-13, were found to be secreted in both the MEF and MEF:hES cell CM. Therefore, it is not unreasonable to predict that one or several of these cytokines may bind to the gp130 receptor and trigger the STAT3 pathway, thus supporting the self-renewal of hES cells.

Taken together, this preliminary study has for the first time clearly revealed intriguing insights into the hES cell in-vitro micro-environment. A new technique has been demonstrated to identify not only what the MEF cells secrete in isolation, but what they secrete in response to the paracrine interactions that occur with the hES cells. Several candidate proteins revealed within this study have roles in differentiation, proliferation and cellular growth. Therefore, future studies will focus on confirming the presence of these candidate proteins as well as assessing their in-vitro biological activity on the hES cell culture system. This study is perhaps the first step towards fully understanding the in-vitro micro-environment of the hES cell and may in fact yield, for the first time, a fully defined, synthetic culture system for hES cells. This development opens avenues for a therapeutically viable tissue source for transplantation.

### Example 2

### Proteomic Analysis of Media Conditioned by Keratinocytes Cultured In-Vitro

This study aimed undertaking a comprehensive examination of the keratinocyte in-vitro micro-environment. In particular, a proteomic approach was adopted to identify the critical factors produced by the feeder cells that are required for keratinocyte growth. Furthermore, a serum-free media as described above, which is fully defined, and has minimal protein content was utilised. The minimal protein content of this serum-free media provides a significant advantage in that it will not “mask” the critical factors secreted by the feeder cells which may be important for supporting keratinocyte cell growth. Additionally, serum-containing media normally requires a pre-processing step before proteomic analysis, such as the “Multiple Affinity Removal System” (MARS) (Agilent Technologies). This MARS immuno-depletion technology involves the removal of high abundant proteins from serum-containing media, which could result in a loss of candidate factors important for the self renewal of primary keratinocytes. Similarly, there is no need to grow the cells in serum-free basal media, an approach routinely adopted in the collection of “conditioned” media. Instead, the media to be analysed was collected from cells cultured in their normal “growth” media; hence they were actively growing, rather than nutrient starved and in a stressed state. Taken together, these features provide an ideal, and a unique position, to identify the critical factors produced in the in-vitro keratinocyte culture microenvironment.

### Methods

### Isolation of Primary Keratinocytes

Primary keratinocytes were isolated from split thickness skin biopsies obtained from breast reductions and abdominoplasties as described by Goberdhan et al. (1993). Briefly, this method involved dissecting the skin biopsy into 0.5 cm² pieces followed by a series of antibiotic wash steps. The skin was then incubated in 0.125% trypsin (Invitrogen, Mulgrave, VIC, Australia) overnight at 4° C. The epidermis was then separated from the dermal layer and the keratinocytes isolated. Keratinocyte cells were then suspended in DMEM (Invitrogen), filtered (100 μm) and pelleted.

### VN: GF-Kc Culture

Freshly isolated keratinocytes were initially cultured in 75 cm² flasks at a density of 2×10⁶ cells and were then seeded at 2×10⁵ cells per 75 cm² flask for subsequent passages. Prior to seeding the keratinocytes, a gamma-irradiated (two doses of 25 Gy) (Australian Red Cross Blood Service, Brisbane, QLD, Australia) mouse i3T3 cell feeder layer was pre-seeded for four hours at 2×10⁶ cells. The feeder layer was then serum-starved for three hours following seeding. The keratinocytes were propagated in VN:GF medium containing: phenol red-free DMEM/HAMS medium (Invitrogen); 0.4 μg/mL hydrocortisone; 0.1 nM cholera toxin; 1.8×10⁻⁴ M adenine; 2×10⁻⁷M triiodo-1-thyronine; 5 μg/mL transferrin; 2×10⁻³ M glutamine (Invitrogen); 1000 IU/mL penicillin/1000 μg/mL streptomycin (Invitrogen); 0.6 μg/mL VN (Promega, Annandale, NSW, Australia); 0.6 μg/mL IGFBP-3 (N109D recombinant mutant) (Auspep, Parkville, VIC, Australia); 0.2 μg/mL IGF-I (GroPep, Adelaide, SA, Australia); and 0.2 μg/mL EGF (Invitrogen) (VN:GF-Kc). The keratinocyte cultures were incubated at 37° C. in 5% carbon dioxide and re-fed with VN:GF-Kc medium every two days. Morphology and marker expression were used to ensure that the keratinocytes used in this experiment were phenotypically similar to those grown using serum. Briefly, this involved probing the cultures with an antibody against keratin 6, a marker expressed by undifferentiated keratinocytes.

### 2-Dimensional Proteomics.

Using methods hereinbefore described in Example 1, two-dimensional liquid chromatography was used to fractionate conditioned media samples and employed a BioLogic Duo-flow high performance liquid chromatography (HPLC) (Bio-rad, Hercules, Calif., USA) for the first dimension separation, while the second stage of the Beckman Coulter's ProteomeLab™ PF 2D platform was utilized for the second dimension separation.

Sample Preparation and LC/MS using LC/ESI/MS and LC-MALDI Analysis.

By using methods as hereinbefore described in Example 1, proteins present in both the feeder cell and feeder cell:keratinocyte conditioned media samples were identified using, two LC/MS procedures were used, LC/ESI/MS and LC-MALDI.

### Sample Preparation and MALDI-TOF-TOF Mass Spectrometry.

Protein peaks were transferred to mass spectrometry plates for TOF-TOF analysis using procedures described in Example 1.

### Database Analysis and Interpretation

The protein score, protein score confidence interval, total ion score (TIS) and total ion score confidence intervals obtained from MS and MS/MS database analysis were used to rank proteins from a list of potential matches.

### Results

Morphology and Expression of Cell Surface Markers Present on the Passage 2 Keratinocytes Propagated using VN:GF-Kc Medium for Proteomic Analysis.

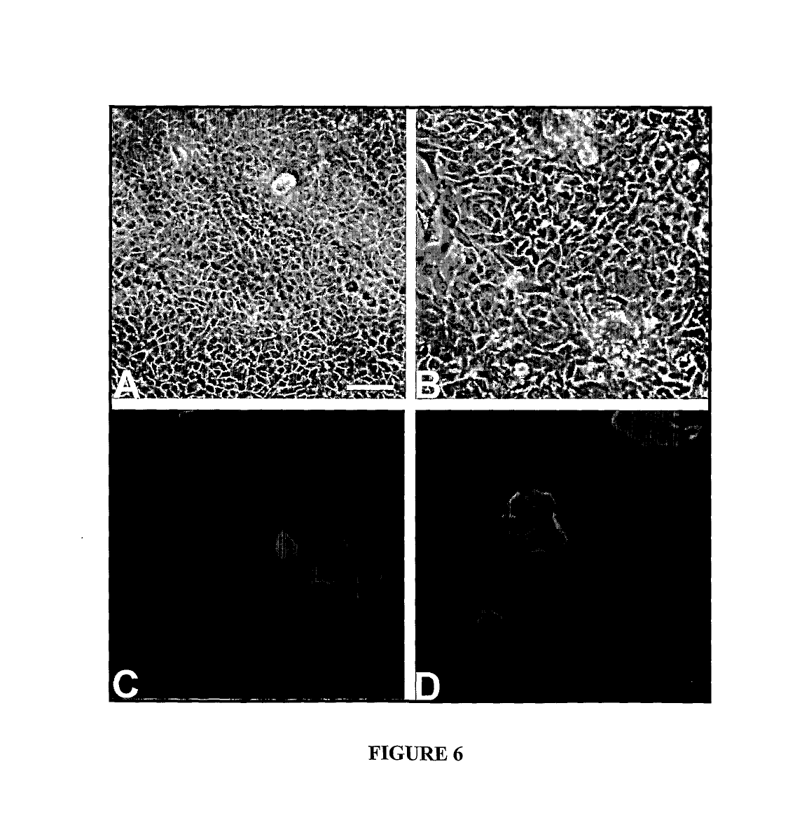

Morphology and marker expression were used to ensure that the conditioned media to be analysed was collected from undifferentiated primary keratinocytes. Presently, there are no definitive assays for determining whether cultured primary keratinocyte cells have maintained an undifferentiated state. However, keratin markers can be used to provide useful information regarding the proliferative state of the cell, and whether or not the cell is a basal keratinocyte. Therefore antibodies that recognise keratin 6 (present in hyper-proliferative keratinocytes), keratin 14 (present in basal cells), and keratin 1/10/11 (present in more differentiated, supra-basal cells, data not shown) were used to assess the differentiation status of the cells cultured for the proteomics study. This analysis revealed that cells propagated using the VN:GF-Kc medium had maintained a normal morphology compared to those grown using serum (FIGS. 6B and A, respectively). Additionally, keratinocytes cultured in the VN:GF-Kc medium continued to express keratin 6 and 14 (FIGS. 6C and D, respectively), thus suggesting these cells have maintained their undifferentiated primary keratinocyte morphology.

Two Dimensional Separation of Conditioned Media Collected from Both Feeder Cells Alone and Feeder Cell:Keratinocyte Cultures.

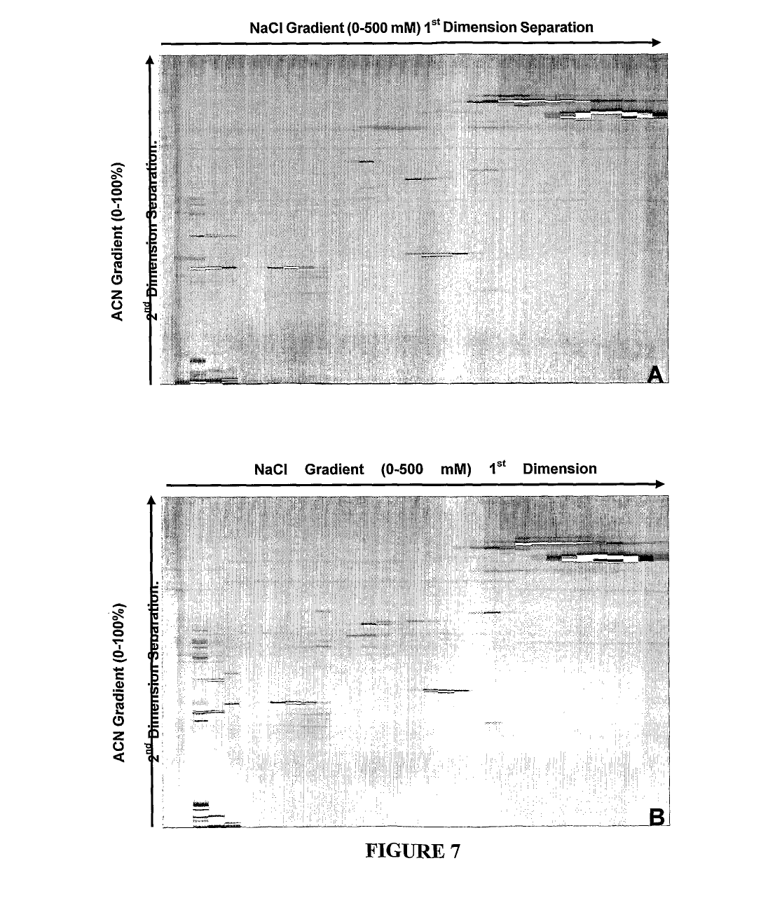

Proteins present in the conditioned media of feeder cells alone and from feeder cell:keratinocyte co-cultures (FIGS. 7A and B, respectively) were separated using a novel form of 2 dimensional liquid chromatography separation. This involved separating proteins via a salt gradient in the first dimension, followed by a second dimension separation using an acetonitrile gradient. The first dimension of the standard Beckman Coulter ProteomeLab was replaced with Bio-rad's Duo-flow HPLC due to poor first dimension resolution of the platform. Proteins were visualised using the ProteoView software. Clearly, there is an increase in the number of distinct protein spots expressed in the feeder cell:keratinocyte culture conditioned media (FIG. 7B), above that found with the feeder cell alone conditioned media. Furthermore, there appear to be observable changes in expression levels between the feeder cells alone conditioned media (FIG. 7A) and that obtained from the feeder cell:keratinocyte cultures (data not shown). Subsequently, 187 protein spots represented in FIG. 7A and 238 protein spots represented in FIG. 7B were isolated, digested and analysed using MALDI TOF-TOF.

### Proteins Identified in the Feeder Cell and the Feeder Cell:Keratinocyte Conditioned Media.

Initially, MALDI-TOF-TOF analysis was performed on the protein spots and did not reveal significant ion scores for the feeder cell alone or the feeder cell:keratinocyte conditioned media (CM). Consequently, the CM was analysed using two liquid chromatography methods; the first involved the QTRAP MS/MS system (LC/ESI/MS) (conducted on fractions from the first dimension separation), while the second utilised LC-MALDI (conducted on the concentrated CM sample) (Table 3 and 4). The Mascot database was then employed to analyse the proteins present in the conditioned media. The LC/ESI/MS and LC-MALDI results were organised into seven major groups; extra-cellular matrix (ECM), membrane, nuclear, secreted, serum-derived and miscellaneous proteins/factors. Additionally, the proteins were categorised using accession number, molecular weight, total score and peptide count. All proteins identified in Tables 3 and 4 are either identified or exhibit extensive homology as determined by ion score. The feeder cell alone results revealed; 12 ECM, 2 growth factors, 17 miscellaneous, 14 membrane, 10 nuclear, 5 secreted, and 3 serum-derived proteins (Table 3). The feeder cell:keratinocyte results revealed; 3 cytoplasmic, 22 ECM, 30 miscellaneous, 21 membrane, 19 nuclear, 9 secreted, and 4 serum-derived proteins. LCMS results were organised via rank which is related to the total ion score (Table 4).

Differences in Expression of Protein Species Found in the Feeder Cell and the Feeder Cell:hES/Keratinocyte Conditioned Media.

Proteins identified using LC-ESI, LC-MALDI and MALDI-TOF-TOF analysis was performed on the liquid fractions obtained from the feeder cell alone or the feeder cell:keratinocyte conditioned media (CM). The Mascot database was employed to analyse the proteins present in these treatments. Potential candidates and proteins of interest were then separated into their respective categories including; Extra-cellular Matrix, Growth Factors and Cytokines, Secreted and Intracellular proteins. There was overlap between proteins in both treatments including: Collagens I, IV and VII; fibronectin I; Laminin V; TGFs alpha and beta; VEGF; Interleukins 1, 10 and 15; Telomerase-binding protein EST1A; and Tra1 homolog. However, unique proteins were also observed in the feeder cell alone treatment including: Wnt-2b, Wnt-12, Collagens V and VI; Bone Morphogenic protein 1 (BMP 1); bFGF; human growth hormone (HGH); FGF 3; Insulin; IGF-I and -II; Interleukin-8; Leukaemia inhibitory factor and Megakaryocyte-CSF. Furthermore, unique proteins were also observed in the feeder cell:keratinocyte treatment including: Fibronectin III; Laminin I and III; nerve growth factor (NGF); hepatocyte growth factor (hgf), PC cell-derived growth factor; platelet-derived growth factor beta (PDGF); Interleukin 4 and 6; PDGF-inducible JE glycoprotein; Follistatin-related protein 5; growth inhibitory factor; Growth differentiation factor 9 and telomerase reverse transcriptase (Table 5).

### Discussion

Many novel technologies involving primary keratinocytes are being developed for the therapeutics industry to aid in the regeneration and healing of skin defects [75,76]. However, technologies used to propagate these cells ex-vivo still require undefined components, such as serum and/or feeder cells, and generally utilise a poorly defined culture system. Whilst a fully defined serum-free technology (VN:GF-Kc) that can support the isolation, establishment and serial propagation of undifferentiated keratinocytes is a step forward, the culture approach still required the use of an irradiated i3T3 feeder layer for successful serial propagation and in-vitro expansion.

It has been demonstrated that irradiated i3T3 feeder cells secrete large quantities of IGFs and ECM proteins [77], as well as a variety of other proteins. Moreover, keratinocytes have also been demonstrated to express the receptors for many growth factors and ECM proteins [78-83]. Indeed, other laboratories have investigated the use of these proteins for the culture of keratinocytes. For example, Dawson et al. (1996) demonstrated that keratinocytes can attach and proliferate in response to VN-coated surfaces [84]. Nevertheless, the most robust culture systems for keratinocytes still require the use of a feeder cell layer [85]. This requirement for a feeder cell layer highlights the importance that the feeder cells have in the culture system. More recently, other groups have demonstrated that other cell types, such as human embryonic stem cells, can be propagated feeder-free using ECM proteins when the culture system is supplemented with conditioned media obtained from MEFs, thus suggesting that the critical component provided by the feeder cells is a soluble factor secreted by the feeder layer [18].

Therefore, the present inventors have hypothesised that novel proteins in conditioned media may be able to be identified using proteomic techniques and that these proteins could potentially be used in conjunction with the VN:GF-Kc medium to support serum-free and feeder cell-free propagation of keratinocytes. Furthermore, given that the VN:GF-Kc media does not contain serum or high abundance proteins such as albumin i.e. it is a low protein content media, a unique position was afforded to identify critical factors important to keratinocyte survival. These factors may normally be masked by these high abundant proteins traditionally incorporated into serum-containing or high protein content media.

To date, most proteomic analysis in this area and related fields, has been conducted on the feeder cell layer alone [24, 25, 86], providing insight into what fibroblasts secrete into the media. However, this research takes this one step further by establishing a system in which secretions triggered by paracrine interactions of the feeder cells with the keratinocytes can also be analysed. To examine this hypothesis it was examined what the feeder cell alone and feeder cell:keratinocyte cultures were secreting into the media. The study of both of these treatments provides a more complete picture of the secreted factors in response to not only the autocrine interactions, but also the paracrine interactions, and gives a greater insight into the optimal in-vitro micro-environment for keratinocytes.

Whilst the system employed here utilized a serum-free VN:GF-Kc medium, several serum-derived proteins were identified in the feeder cell alone and feeder cell:keratinocyte treatments; namely, bovine serum albumin, fetuin, and members of the transferrin family. These proteins are all common constituents of the serum and supplements commonly added to media for the propagation of feeder cells and keratinocytes. The presence of these proteins in this analysis indicates that whilst the VN:GF-Kc serum-free medium was used for this proteomic investigation, serum products were carried over from the original expansion of the fibroblast cells, despite extensive washing and serum-starvation steps. In addition, the data suggests that serum-derived proteins were carried over from the donor patient's skin during the keratinocyte isolation, as keratinocytes themselves were isolated and cultured entirely serum-free. Furthermore, several intra-cellular proteins were observed within both treatments of this study. The presence of these proteins is most likely due to the cells lysing, hence leaking their intracellular contents into the culture system. Whilst these intracellular proteins were not the prime focus of this study, some of the proteins identified warrant further investigation such as telomerase reverse transcriptase, telomerase binding protein, c-myc, and Tra1. It is also important to note here that several proteins were omitted from tables 3 and 4 due to the fact that they could not be identified i.e. hypothetical proteins, unknown proteins, and proteins with no known function.

The analysis of the feeder cell alone conditioned medium (FIG. 7A, and Table 3) and the feeder cell:keratinocyte culture conditioned medium (FIG. 7B, and Table 4), revealed several proteins important for the survival of primary keratinocytes. Several ECM proteins were identified and include; Collagen I, V, VI, and VII, Fibronectin 1 and 3, Lamb3, Laminin alpha 1, 3, 5, and Tenascin X (Tables 3 and 4). Importantly, these ECM proteins are found in-vivo in the extra-cellular matrix of the epidermis and dermis [87]. Furthermore, these proteins are commonly involved in the attachment, migration and or proliferation of keratinocytes, and also have been proposed to have roles in wound healing [88-91].

Research groups involved in the development of serum-free and feeder cell-free culture methods for hES cells have recently commenced exploring the use of ECM proteins, such as those mentioned, in their culture systems. For example, laminin was demonstrated to replace the need for a feeder cell layer when grown in the presence of mouse embryonic fibroblast (MEF) conditioned medium [18] or with knock-out serum replacement (KSR)+Activin-A [21]. Moreover, Amit et al. (2006) discovered a method to propagate these cells using a fibronectin matrix in conjunction with a range of growth factors including, transforming growth factor β1 (TGF β1), leukaemia inhibitory factor (LIF) and basic fibroblast growth factor (bFGF) [20]. Due to the fact that the culture of primary keratinocytes is analogous to hES cell culture, it is likely that these ECM protein-based technologies can be translated to the culture of keratinocytes. Interestingly, all the ECM technologies developed for the propagation of keratinocyte and hES cells thus far, also involve the use of some form of mitogen.

The results reported herein demonstrated that several growth factors and mitogens were present in the conditioned medium including IGF-I, IGF-II, insulin, transforming growth factors (TGF) α and β, platelet-derived growth factor (PDGF) and bFGF, all of these being present in the conditioned media of the two treatments (Tables 3 and 4). Insulin is a critical component in many mammalian cell culture media and has been incorporated into the culture of keratinocytes for some time now. Usually insulin is present in these keratinocyte culture media at high concentrations, however, we recently demonstrated that low concentrations of IGF-I can replace the need for insulin [26, 41]. Indeed, it has been reported that when insulin is present at high concentrations its growth stimulating effects are in fact mediated by the IGF-I receptor [92], hence the ability to replace insulin with IGF-I is not surprising. Similarly, earlier studies conducted within our group revealed that IGF-I and IGF-II when used in conjunction with VN caused mitogenic affects in hES cells. Moreover, bFGF, TGF-beta and PDGF, are heparin binding growth factors that have been demonstrated to enhance the proliferation and self renewal of feeder cell dependent hES cells [16, 23, 44, 45]. Furthermore, the TGF proteins have been demonstrated to enhance migration (Li et al. 2006) and proliferation of epidermal and keratinocyte cells [93, 94] and thus have been proposed as potentially being effective in mediating wound healing events [95]. Interestingly, these heparin-binding growth factors appear to be able to bind to VN through its heparin binding domain [26, 42]. Thus, the growth factors identified in this proteomic analysis all have roles related to keratinocyte growth and may well prove to be useful in conjunction with the VN:GF-Kc medium in providing a serum-free, feeder-free media for the in-vitro expansion of transplantable cells for use in clinical therapies.

Additionally, Wnt-12 and human growth hormone present in feeder CM, and growth differentiation factor-9 (GDF-9) and PC-derived growth factor (PC-DGF) present in the feeder cell:keratinocyte CM were also identified (Table 5). To date not much is known on the effects of these proteins on the growth and survival of hES cells. However, the Wnt pathway and certain Wnt proteins have been demonstrated to maintain hES cells in a state of self-renewal [56]. Human growth hormone may also play an important role in the self-renewal of hES cells by activating the JAK/STAT pathway [66, 72]. Furthermore, PC derived growth factor is shown to be widely expressed during embryonic development and has demonstrated a role in proliferation in cells such as 3T3 fibroblasts [71]. Additionally, GDF-9 has been demonstrated to activate SMAD-2/3 signaling [73], which is important for maintaining the hES cells in an undifferentiated state [74]. These factors may therefore be important for other cells that are cultured in a similar manner to hES cells i.e primary keratinocyte cells.

In addition to the proteins discussed above, telomerase reverse transcriptase (TERT) telomerase-binding protein (EST1A), Follistatin-like 5, and tumor rejection antigen1 (Tra1) homolog, were also expressed in the conditioned of both treatments (Tables 3 and 4). The telomerase-binding protein is involved in telomere replication in-vitro via human telomerase reverse transcriptase. Interestingly, a down regulation in hTERT or telomerase expression is linked to embryonic stem cell differentiation [62]. Therefore, if this protein can be induced, directly or indirectly, in the culture of keratinocytes, it may facilitate the long term propagation of primary keratinocytes. Another nuclear protein that may be of interest is the Tra1 homolog which has a central role in c-Myc transcription activation, and also participates in cell transformation. Furthermore, c-Myc has been demonstrated to be important in the activation and regulation of hTERT [63]. The secreted protein, follistatin-like 5, was also present in the conditioned media examined. Notably, the follistatin-like domain present in this protein has been implicated in the inactivation of activin-A and TGF-β [96, 97], two proteins which have been demonstrated to be important for the self renewal of human embryonic stem cells [16]. Taken together, this data suggests that these proteins may also play an important role in maintaining the undifferentiated status of other primitive cells, such as primary keratinocytes.

In summary, the proteomic study reported here has revealed the expression of many proteins from both the feeder cells alone and the feeder cell:keratinocyte culture treatments. In light of the paracrine relationship which exists between the dermal fibroblasts and keratinocytes [98, 99], the study here identified not only what the feeder cells are secreting in isolation, but what they and the keratinocytes secrete due to their paracrine interactions. Ideally, it would have been of great benefit to also examine media conditioned by keratinocytes alone to determine what these cells secrete when cultivated without feeder cells. However, this highlights the key point of this investigation, i.e. keratinocyte cells grow poorly in the absence of a feeder cell layer. The aforementioned data has provided intriguing preliminary insights into the in-vitro micro-environment of primary keratinocytes and has provided useful initial information on candidate proteins that may be used in conjunction with the serum-free medium.

### Example 3

### Feeder- and Serum-Free Growth of hES Cells

hES cells were grown and tested with the following medium formulation 1 ug/mL IGF-I/1-64VN chimeric protein, 0.1 ug/mL bFGF, 35 ng/mL Activin-A and 40 μg/mL laminin.

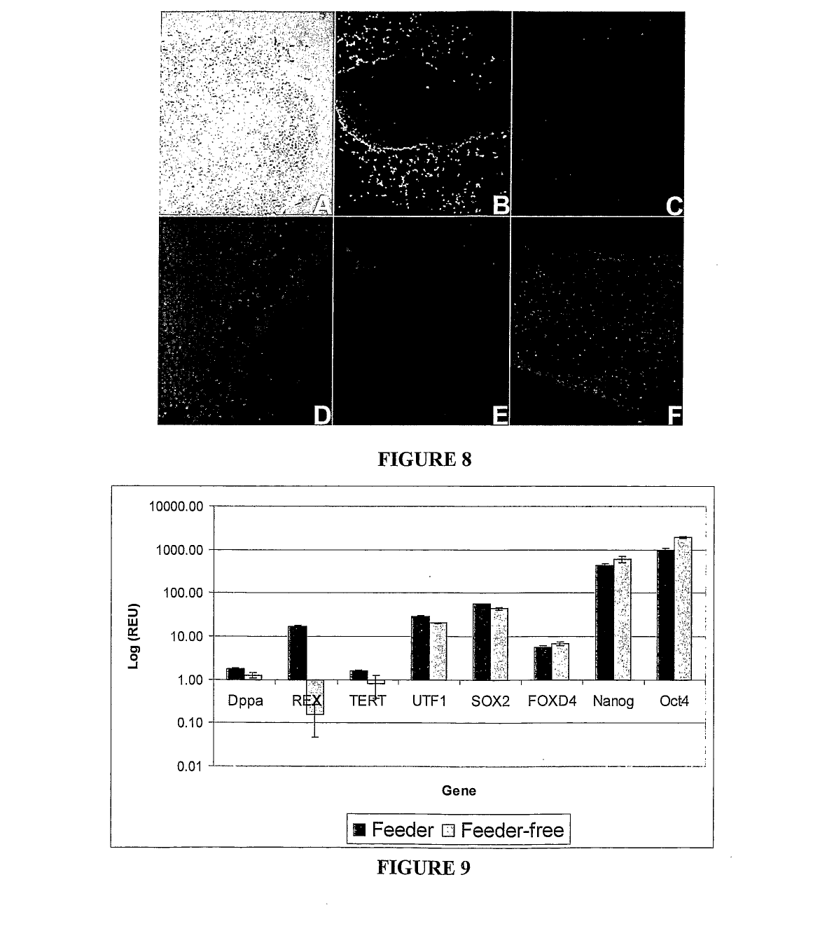

Immunofluorescence (IF) was conducted using antibodies directed towards Oct4, TRA1-60, SSEA-4, SSEA-1 antigens. The IF studies (in FIG. 8) demonstrated expression of Oct4, TRA1-60 and SSEA-4 but only low expression of SSEA-1. The hES cells also presented with a large nucleus to cytoplasmic ratio indicative of a hES phenotype.

Rex1 is an anomaly this result demonstrates massive down regulation within our culture system. However, when Oct4 and Nanog were examined these amplicons revealed almost a 2 fold increase in expression within our culture system (see FIG. 9).

These data taken together suggest that the system described can indeed maintain these cells in an “undifferentiated state”.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

1. Thomson, J. A., J. Itskovitz-Eldor, S. S. Shapiro, M. A. Waknitz, J. J. Swiergiel, V. S. Marshall, and J. M. Jones. (1998). Embryonic stem cell lines derived from human blastocysts. Science 282: 1145-7.
2. Reubinoff, B. E., M. F. Pera, C. Y. Fong, A. Trounson, and A. Bongso. (2000). Embryonic stem cell lines from human blastocysts: somatic differentiation in vitro. Nature Biotechnology 18: 399-404.
3. Levy, Y. S., M. Stroomza, E. Melamed, and D. Offen. (2004). Embryonic and adult stem cells as a source for cell therapy in Parkinson's disease. J Mol Neuroscience 24: 353-86.
4. Tutter, A. V., G. A. Baltus, and S. Kadam. (2006). Embryonic stem cells: a great hope for a new era of medicine. Current Opinion Drug Discovery Development 9: 169-75.
5. Keirstead, H. S., G. Nistor, G. Bernal, M. Totoiu, F. Cloutier, K. Sharp, and O. Steward. (2005). Human embryonic stem cell-derived oligodendrocyte progenitor cell transplants remyelinate and restore locomotion after spinal cord injury. Journal of Neuroscience 25: 4694-705.
6. Rohwedel, J., K. Guan, C. Hegert, and A. M. Wobus. (2001). Embryonic stem cells as an in vitro model for mutagenicity, cytotoxicity and embryotoxicity studies: present state and future prospects. Toxicology In-Vitro 15: 741-53.
7. Henderson, J. K., J. S. Draper, H. S. Baillie, S. Fishel, J. A. Thomson, H. Moore, and P. W. Andrews. (2002). Preimplantation human embryos and embryonic stem cells show comparable expression of stage-specific embryonic antigens. Stem Cells 20: 329-37.
8. Schick, B. P., H. C. Ho, K. C. Brodbeck, C. W. Wrigley, and J. Klimas. (2003). Serglycin proteoglycan expression and synthesis in embryonic stem cells. Biochimica et biophysica acta 1593: 259-67.
9. Amit, M., V. Margulets, H. Segev, K. Shariki, I. Laevsky, R. Coleman, and J. Itskovitz-Eldor. (2003). Human feeder layers for human embryonic stem cells. Biology of Reproduction 68: 2150-6.
10. Hovatta, O., M. Mikkola, K. Gertow, A. M. Stromberg, J. Inzunza, J. Hreinsson, B. Rozell, E. Blennow, M. Andang, and L. Ahrlund-Richter. (2003). A culture system using human foreskin fibroblasts as feeder cells allows production of human embryonic stem cells. Human Reproduction 18: 1404-9.
11. Cheng, L., H. Hammond, Z. Ye, X. Zhan, and G. Dravid. (2003). Human adult marrow cells support prolonged expansion of human embryonic stem cells in culture. Stem Cells 21: 131-42.
12. Carpenter, M. K., E. S. Rosier, G. J. Fisk, R. Brandenberger, X. Ares, T. Miura, M. Lucero, and M. S. Rao. (2004). Properties of four human embryonic stem cell lines maintained in a feeder-free culture system. Developmental Dynamics 229: 243-58.
13. Draper, J. S., K. Smith, P. Gokhale, H. D. Moore, E. Maltby, J. Johnson, L. Meisner, T. P. Zwaka, J. A. Thomson, and P. W. Andrews. (2004). Recurrent gain of chromosomes 17q and 12 in cultured human embryonic stem cells. Nature Biotechnology 22: 53-4.
14. Rosler, E. S., G. J. Fisk, X. Ares, J. Irving, T. Miura, M. S. Rao, and M. K. Carpenter. (2004). Long-term culture of human embryonic stem cells in feeder-free conditions. Developmental Dynamics 229: 259-74.
15. Xu, C., E. Rosier, J. Jiang, J. S. Lebkowski, J. D. Gold, C. O'Sullivan, K. Delavan-Boorsma, M. Mok, A. Bronstein, and M. K. Carpenter. (2005). Basic fibroblast growth factor supports undifferentiated human embryonic stem cell growth without conditioned medium. Stem Cells 23: 315-23.
16. James, D., A. J. Levine, D. Besser, and A. Hemmati-Brivanlou. (2005). TGFbeta/activin/nodal signalling is necessary for the maintenance of pluripotency in human embryonic stem cells. Development 132: 1273-82.
17. Ludwig, T. E., M. E. Levenstein, J. M. Jones, W. T. Berggren, E. R. Mitchen, J. L. Frane, L. J. Crandall, C. A. Daigh, K. R. Conard, M. S. Piekarczyk, R. A. Llanas, and J. A. Thomson. (2006). Derivation of human embryonic stem cells in defined conditions. Nature Biotechnology 24: 185-7.
18. Xu, C., M. S. Inokuma, J. Denham, K. Golds, P. Kundu, J. D. Gold, and M. K. Carpenter. (2001). Feeder-free growth of undifferentiated human embryonic stem cells. Nature Biotechnology 19: 971-4.
19. Koivisto, H., M. Hyvarinen, A. M. Stromberg, J. Inzunza, E. Matilainen, M. Mikkola, O. Hovatta, and H. Teerijoki. (2004). Cultures of human embryonic stem cells: serum replacement medium or serum-containing media and the effect of basic fibroblast growth factor. Reproductive Biomedicine Online 9: 330-7.
20. Amit, M. and J. Itskovitz-Eldor. (2006). Feeder-free culture of human embryonic stem cells. Methods in Enzymology 420: 37-49.
21. Beattie, G. M., A. D. Lopez, N. Bucay, A. Hinton, M. T. Firpo, C. C. King, and A. Hayek. (2005). Activin A maintains pluripotency of human embryonic stem cells in the absence of feeder layers. Stem Cells 23: 489-95.
22. Xu, R. H., R. M. Peck, D. S. Li, X. Feng, T. Ludwig, and J. A. Thomson. (2005). Basic FGF and suppression of BMP signaling sustain undifferentiated proliferation of human ES cells. Nature Methods 2: 185-90.
23. Pebay, A., R. C. Wong, S. M. Pitson, E. J. Wolvetang, G. S. Peh, A. Filipczyk, K. L. Koh, I. Tellis, L. T. Nguyen, and M. F. Pera. (2005). Essential roles of sphingosine-1-phosphate and platelet-derived growth factor in the maintenance of human embryonic stem cells. Stem Cells 23: 1541-8.
24. Prowse, A. B., L. R. McQuade, K. J. Bryant, D. D. Van Dyk, B. E. Tuch, and P. P. Gray. (2005). A proteome analysis of conditioned media from human neonatal fibroblasts used in the maintenance of human embryonic stem cells. Proteomics 5: 978-89.
25. Lim, J. W. and A. Bodnar. (2002). Proteome analysis of conditioned medium from mouse embryonic fibroblast feeder layers which support the growth of human embryonic stem cells. Proteomics 2: 1187-203.
26. Hollier, B., D. G. Harkin, D. Leavesley, and Z. Upton. (2005). Responses of keratinocytes to substrate-bound vitronectin: growth factor complexes. Experimental Cell Research 305: 221-32.
27. Kricker, J. A., C. L. Towne, S. M. Firth, A. C. Herington, and Z. Upton. (2003). Structural and functional evidence for the interaction of insulin-like growth factors (IGFs) and IGF binding proteins with vitronectin. Endocrinology 144: 2807-15.
28. Upton, Z. and J. A. Kricker, International Patent. WO 02/24219 A1. 2002.
29. zur Nieden, N. I., L. J. Ruf, G. Kempka, H. Hildebrand, and H. J. Ahr. (2001). Molecular markers in embryonic stem cells. Toxicology In-Vitro 15: 455-61.
30. Martin, M. J., A. Muotri, F. Gage, and A. Varki. (2005). Human embryonic stem cells express an immunogenic nonhuman sialic acid. Nature Medicine 11: 228-32.
31. Richards, S., D. Leavesley, and Z. Upton. (unpublished data). Serum-free growth of human embryonic stem cells.
32. Barreca, A., M. De Luca, P. Del Monte, S. Bondanza, G. Damonte, G. Cariola, E. Di Marco, G. Giordano, R. Cancedda, and F. Minuto. (1992). In vitro paracrine regulation of human keratinocyte growth by fibroblast-derived insulin-like growth factors. Journal of Cellular Physiology 151: 262-8.
33. Draper, J. S., H. D. Moore, L. N. Ruban, P. J. Gokhale, and P. W. Andrews. (2004). Culture and characterization of human embryonic stem cells. Stem Cells and Development 13: 325-36.
34. Richards, M., C. Y. Fong, W. K. Chan, P. C. Wong, and A. Bongso. (2002). Human feeders support prolonged undifferentiated growth of human inner cell masses and embryonic stem cells. Nature Biotechnology 20: 933-6.
35. Lawler, J., M. Sunday, V. Thibert, M. Duquette, E. L. George, H. Rayburn, and R. O. Hynes. (1998). Thrombospondin-1 is required for normal murine pulmonary homeostasis and its absence causes pneumonia Journal of Clinical Investigation 101: 982-92.
36. Hochegger, K., S. Knight, C. Hugo, G. Mayer, J. Lawler, T. N. Mayadas, and A. R. Rosenkranz. (2004). Role of thrombospondin-1 in the autologous phase of an accelerated model of anti-glomerular basement membrane glomerulonephritis. Nephron Experimental Nephrology 96: e31-8.
37. Hugo, C., R. Pichler, R. Meek, K. Gordon, T. Kyriakides, J. Floege, P. Bornstein, W. G. Couser, and R. J. Johnson. (1995). Thrombospondin 1 is expressed by proliferating mesangial cells and is up-regulated by PDGF and bFGF in vivo. Kidney International 48: 1846-56.
38. Tada, H. and S. Isogai. (1998). The fibronectin production is increased by thrombospondin via activation of TGF-beta in cultured human mesangial cells. Nephron 79: 38-43.
39. Lu, J., R. Hou, C. J. Booth, S. H. Yang, and M. Snyder. (2006). Defined culture conditions of human embryonic stem cells. Proceedings of the National Academy of Sciences of the United States of America 103: 5688-93.
40. Upton, Z., H. Webb, K. Hale, C. A. Yandell, J. P. McMurtry, G. L. Francis, and F. J. Ballard. (1999). Identification of vitronectin as a novel insulin-like growth factor-II binding protein. Endocrinology 140: 2928-31.
41. Hyde, C., B. Hollier, A. Anderson, D. Harkin, and Z. Upton. (2004). Insulin-like growth factors (IGF) and IGF-binding proteins bound to vitronectin enhance keratinocyte protein synthesis and migration. Journal of Investigative Dermatology 122: 1198-206.
42. Schoppet, M., T. Chavakis, N. Al-Fakhri, S. M. Kanse, and K. T. Preissner. 2002). Molecular interactions and functional interference between vitronectin and transforming growth factor-beta Laboratory Investigations 82: 37-46.
43. Schuldiner, M., O. Yanuka, J. Itskovitz-Eldor, D. A. Melton, and N. Benvenisty. (2000). Effects of eight growth factors on the differentiation of cells derived from human embryonic stem cells. Proceedings of the National Academy of Sciences of the United States of America 97: 11307-12.
44. Liu, Y., Z. Song, Y. Zhao, H. Qin, J. Cai, H. Zhang, T. Yu, S. Jiang, G. Wang, M. Ding, and H. Deng. (2006). A novel chemical-defined medium with bFGF and N2B27 supplements supports undifferentiated growth in human embryonic stem cells. Biochemical and Biophysical Research Communications 346: 131-9.
45. Wang, G., H. Zhang, Y. Zhao, J. Li, J. Cai, P. Wang, S. Meng, J. Feng, C. Miao, M. Ding, D. Li, and H. Deng. (2005). Noggin and bFGF cooperate to maintain the pluripotency of human embryonic stem cells in the absence of feeder layers. Biochemical and Biophysical Research Communications 330: 934-42.
46. Xu, R. H., X. Chen, D. S. Li, R. Li, G. C. Addicks, C. Glennon, T. P. Zwaka, and J. A. Thomson. (2002). BMP4 initiates human embryonic stem cell differentiation to trophoblast Nature Biotechnology 20: 1261-4.
47. Gerrard, L., L. Rodgers, and W. Cui. (2005). Differentiation of human embryonic stem cells to neural lineages in adherent culture by blocking bone morphogenetic protein signaling. Stem Cells 23: 1234-41.
48. Otsuka, F., R. K. Moore, S. Iemura, N. Ueno, and S. Shimasaki. (2001). Follistatin inhibits the function of the oocyte-derived factor BMP-15. Biochemical and Biophysical Research Communications 289: 961-6.
49. Xiao, L., X. Yuan, and S. J. Sharkis. (2006). Activin A maintains self-renewal and regulates fibroblast growth factor, Wnt, and bone morphogenic protein pathways in human embryonic stem cells. Stem Cells 24: 1476-86.
50. Mummery, C. L., M. van Rooyen, M. Bracke, J. van den Eijnden-van Raaij, E. J. van Zoelen, and K. Alitalo. (1993). Fibroblast growth factor-mediated growth regulation and receptor expression in embryonal carcinoma and embryonic stem cells and human germ cell tumours. Biochemical and Biophysical Research Communications 191: 188-95.
51. Reilly, J. F., G. Mickey, and P. A. Maher. (2000). Association of fibroblast growth factor receptor 1 with the adaptor protein Grb14. Characterization of a new receptor binding partner. Journal of Biology Chemistry 275: 7771-8.
52. Cailliau, K., V. Le Marcis, V. Bereziat, D. Perdereau, B. Cariou, J. P. Vilain, A. F. Burnol, and E. Browaeys-Poly. (2003). Inhibition of FGF receptor signalling in Xenopus oocytes: differential effect of Grb7, Grb10 and Grb14. FEBS Letters 548: 43-8.
53. Hemming, R., R. Agatep, K. Badiani, K. Wyant, G. Arthur, R. D. Gietz, and B. Triggs-Raine. (2001). Human growth factor receptor bound 14 binds the activated insulin receptor and alters the insulin-stimulated tyrosine phosphorylation levels of multiple proteins. Biochemistry and Cell Biology 79: 21-32.
54. Kasus-Jacobi, A., D. Perdereau, C. Auzan, E. Clauser, E. Van Obberghen, F. Mauvais-Jarvis, J. Girard, and A. F. Burnol. (1998). Identification of the rat adapter Grb14 as an inhibitor of insulin actions. Journal of Biology Chemistry 273: 26026-35.
55. Depetris, R. S., J. Hu, I. Gimpelevich, L. J. Holt, R. J. Daly, and S. R. Hubbard. (2005). Structural basis for inhibition of the insulin receptor by the adaptor protein Grb14. Molecular Cell 20: 325-33.
56. Dravid, G., Z. Ye, H. Hammond, G. Chen, A. Pyle, P. Donovan, X. Yu, and L. Cheng. (2005). Defining the role of Wnt/beta-catenin signaling in the survival, proliferation, and self-renewal of human embryonic stem cells. Stem Cells 23: 1489-501.
57. Liu, H., O. Mohamed, D. Dufort, and V. A. Wallace. (2003). Characterization of Wnt signaling components and activation of the Wnt canonical pathway in the murine retina. Developmental Dynamics 227: 323-34.
58. Kawano, Y. and R. Kypta. (2003). Secreted antagonists of the Wnt signalling pathway. Journal of Cell Science 116: 2627-34.
59. Peters, J. M., R. M. McKay, J. P. McKay, and J. M. Graff. (1999). Casein kinase I transduces Wnt signals. Nature 401: 345-50.
60. Takada, R., H. Hijikata, H. Kondoh, and S. Takada. (2005). Analysis of combinatorial effects of Wnts and Frizzleds on beta-catenin/armadillo stabilization and Dishevelled phosphorylation. Genes Cells 10: 919-28.
61. Song, M. R., R. Shirasaki, C. L. Cai, E. C. Ruiz, S. M. Evans, S. K. Lee, and S. L. Pfaff. (2006). T-Box transcription factor Tbx20 regulates a genetic program for cranial motor neuron cell body migration. Development 133: 4945-55.
62. Wang, S., C. Hu, and J. Zhu. (2007). Transcriptional silencing of a novel hTERT reporter locus during in vitro differentiation of mouse embryonic stem cells. Molecular Biology of the Cell 18: 669-77.
63. Chen, Z., Z. Wu, X. Guo, and M. Jiang. (2006). [Expression of telomerase reverse transcriptase and its relationship with expression of c-myc in laryngeal squamous cell carcinomas]. Lin Chuang Er Bi Yan Hou Ke Za Zhi 20: 963-6.
64. Sidis, Y., D. V. Tortoriello, W. E. Holmes, Y. Pan, H. T. Keutmann, and A. L. Schneyer. (2002). Follistatin-related protein and follistatin differentially neutralize endogenous vs. exogenous activin. Endocrinology 143: 1613-24.
65. Li, Y., J. Fan, M. Chen, W. Li, and D. T. Woodley. (2006). Transforming growth factor-alpha: a major human serum factor that promotes human keratinocyte migration. Journal of Investigative Dermatology 126: 2096-105.
66. Matsuda, T., T. Nakamura, K. Nakao, T. Arai, M. Katsuki, T. Heike, and T. Yokota. (1999). STAT3 activation is sufficient to maintain an undifferentiated state of mouse embryonic stem cells. Embo Journal 18: 4261-9.
67. Chung, C. D., J. Liao, B. Liu, X. Rao, P. Jay, P. Berta, and K. Shuai. (1997). Specific inhibition of Stat3 signal transduction by PIAS3. Science 278: 1803-5.
68. Ruff-Jamison, S., Z. Zhong, Z. Wen, K. Chen, J. E. Darnell, Jr., and S. Cohen. (1994). Epidermal growth factor and lipopolysaccharide activate Stat3 transcription factor in mouse liver. Journal of Biology Chemistry 269: 21933-5.
69. Nichols, J., I. Chambers, and A. Smith. (1994). Derivation of germline competent embryonic stem cells with a combination of interleukin-6 and soluble interleukin-6 receptor. Experimental Cell Research 215: 237-9.
70. Humphrey, R. K., G. M. Beattie, A. D. Lopez, N. Bucay, C. C. King, M. T. Firpo, S. Rose-John, and A. Hayek. (2004). Maintenance of pluripotency in human embryonic stem cells is STAT3 independent. Stem Cells 22: 522-30.
71. Xia, X. and G. Serrero, Identification of cell surface binding sites for PC-cell-derived growth factor, PCDGF, (epithelin/granulin precursor) on epithelial cells and fibroblasts. Biochem Biophys Res Commun, 1998. 245(2): p. 539-43.
72. Strous, G. J., et al., Growth hormone-induced signal tranduction depends on an intact ubiquitin system. J Biol Chem, 1997. 272(1): p. 40-3.
73. Kaivo-Oja, N., et al., Growth differentiation factor-9 induces Smad2 activation and inhibin B production in cultured human granulosa-luteal cells. J Clin Endocrinol Metab, 2003. 88(2): p. 755-62.
74. Vallier, L., M. Alexander, and R. A. Pedersen, Activin/Nodal and FGF pathways cooperate to maintain pluripotency of human embryonic stem cells. J Cell Sci, 2005. 118(Pt 19): p. 4495-509.
75. Harkin, D. G., R. A. Dawson and Z. Upton (2006). “Optimized delivery of skin keratinocytes by aerosolization and suspension in fibrin tissue adhesive.” Wound Repair Regen 14(3): 354-63.
76. Sun, T., D. Norton, A. J. Ryan, S. MacNeil and J. W. Haycock (2007). “Investigation of fibroblast and keratinocyte cell-scaffold interactions using a novel 3D cell culture system.” J Mater Sci Mater Med 18(2): 321-8.
77. Barreca, A., M. De Luca, P. Del Monte, S. Bondanza, G. Damonte, G. Cariola, E. Di Marco, G. Giordano, R. Cancedda and F. Minuto (1992). “In vitro paracrine regulation of human keratinocyte growth by fibroblast-derived insulin-like growth factors.” J Cell Physiol 151(2): 262-8.
78. Adams, J. C. and F. M. Watt (1991). “Expression of beta 1, beta 3, beta 4, and beta 5 integrins by human epidermal keratinocytes and non-differentiating keratinocytes.” J Cell Biol 115(3): 829-41.
79. Haase, I., R. Evans, R. Pofahl and F. M. Watt (2003). “Regulation of keratinocyte shape, migration and wound epithelialization by IGF-1- and EGF-dependent signalling pathways.” J Cell Sci 116(Pt 15): 3227-38.
80. Rodeck, U., M. Jost, J. DuHadaway, C. Kari, P. J. Jensen, B. Risse and D. L. Ewert (1997). “Regulation of Bcl-xL expression in human keratinocytes by cell-substratum adhesion and the epidermal growth factor receptor.” Proc Natl Acad Sci USA 94(10): 5067-72.
81. Stoll, S., W. Garner and J. Elder (1997). “Heparin-binding ligands mediate autocrine epidermal growth factor receptor activation In skin organ culture.” J Clin Invest 100(5): 1271-81.
82. Watt, F. M. and P. H. Jones (1993). “Expression and function of the keratinocyte integrins.” Dev Suppl: 185-92.
83. Watt, F. M., M. D. Kubler, N. A. Hotchin, L. J. Nicholson and J. C. Adams (1993). “Regulation of keratinocyte terminal differentiation by integrin-extracellular matrix interactions.” J Cell Sci 106 (Pt 1): 175-82.
84. Dawson, R. A., N. J. Goberdhan, E. Freedlander and S. MacNeil (1996). “Influence of extracellular matrix proteins on human keratinocyte attachment, proliferation and transfer to a dermal wound model.” Burns 22(2): 93-100.
85. Huang, Y. C., T. W. Wang, J. S. Sun and F. H. Lin (2006). “Investigation of mitomycin-C-treated fibroblasts in 3-D collagen gel and conditioned medium for keratinocyte proliferation.” Artif Organs 30(3): 150-9.
86. Boraldi, F., L. Bini, S. Liberatori, A. Armini, V. Pallini, R. Tiozzo, I. Pasquali-Ronchetti and D. Quaglino (2003). “Proteome analysis of dermal fibroblasts cultured in vitro from human healthy subjects of different ages.” Proteomics 3(6): 917-29.
87. Marionnet, C., C. Pierrard, C. Vioux-Chagnoleau, J. Sok, D. Asselineau and F. Bernerd (2006). “Interactions between fibroblasts and keratinocytes in morphogenesis of dermal epidermal junction in a model of reconstructed skin.” J Invest Dermatol 126(5): 971-9.
88. Rho, K. S., L. Jeong, G. Lee, B. M. Seo, Y. J. Park, S. D. Hong, S. Roh, J. J. Cho, W. H. Park and B. M. Min (2006). “Electrospinning of collagen nanofibers: effects on the behavior of normal human keratinocytes and early-stage wound healing.” Biomaterials 27(8): 1452-61.
89. Schneider, H., C. Muhle and F. Pacho (2006). “Biological function of laminin-5 and pathogenic impact of its deficiency.” Eur J Cell Biol.
90. Spichkina, O. G., N. V. Kalmykova, L. V. Kukhareva, I. V. Voronkina, M. I. Blinova and G. P. Pinaev (2006). “[Isolation of human basal keratinocytes by selective adhesion to extracellular matrix proteins].” Tsitologiia 48(10): 841-7.
91. Hartwig, B., B. Borm, H. Schneider, M. J. Arin, G. Kirfel and V. Herzog (2007). “Laminin-5-deficient human keratinocytes: defective adhesion results in a saltatory and inefficient mode of migration.” Exp Cell Res 313(8): 1575-87.
92. Dupont, J. and D. LeRoith (2001). “Insulin and insulin-like growth factor I receptors: similarities and differences in signal transduction.” Horm Res 55 Suppl 2: 22-6.
93. Fortunel, N. O., J. A. Hatzfeld, P. A. Rosemary, C. Ferraris, M. N. Monier, V. Haydont, J. Longuet, B. Brethon, B. Lim, I. Castiel, R. Schmidt and A. Hatzfeld (2003). “Long-term expansion of human functional epidermal precursor cells: promotion of extensive amplification by low TGF-beta1 concentrations.” J Cell Sci 116(Pt 19): 4043-52.
94. Sawamura, D., S. Ina, M. Goto, M. Akiyama and H. Shimizu (2004). “In vivo transfer of TGF-alpha and beta genes to keratinocytes.” J Dermatol Sci 34(3): 234-6.
95. Sun, T., M. Higham, C. Layton, J. Haycock, R. Short and S. MacNeil (2004). “Developments in xenobiotic-free culture of human keratinocytes for clinical use.” Wound Repair Regen 12(6): 626-34.
96. Schneyer, A., A. Schoen, A. Quigg and Y. Sidis (2003). “Differential binding and neutralization of activins A and B by follistatin and follistatin like-3 (FSTL-3/FSRP/FLRG).” Endocrinology 144(5): 1671-4.
97. Schneyer, A., Y. Sidis, Y. Xia, S. Saito, E. del Re, H. Y. Lin and H. Keutmann (2004). “Differential actions of follistatin and follistatin-like 3.” Mol Cell Endocrinol 225(1-2): 25-8.
98. Maas-Szabowski, N., A. Shimotoyodome and N. E. Fusenig (1999). “Keratinocyte growth regulation in fibroblast cocultures via a double paracrine mechanism.” J Cell Sci 112 (Pt 12): 1843-53.
99. Werner, S. and H. Smola (2001). “Paracrine regulation of keratinocyte proliferation and differentiation.” Trends Cell Biol 11(4): 143-6.

### TABLES

TABLE 1
Proteins identified from feeder cell conditioned media using 4000 MALDI-
TOF-TOF system, QTRAP MS/MS and, LC-MALDI.

| | | | MALDI | QTRAP | |
|---|---|---|---|---|---|
| | | | MS-MS | MS-MS | LC- |
| | | MW | Protein | Mowse | MALDI |
| Protein | Accession Number | (kDa) | score | Score | Ion Score |
| Extra Cellular Matrix | | | | | |
| Collagen alpha-1(I) chain [Precursor] | Q63079 | 138 | | 64 | |
| Collagen alpha-1(V) chain [Precursor] | BAA14323 | 184 | | 41 | |
| Collagen alpha-3(VI) chain [Precursor] | CAB60731 | 549 | | 52 | |
| Collagen alpha-1(VII) chain [Precursor] | AAA58965 | 293 | | 59 | |
| Collagen alpha-1(XII) chain [Precursor] | AAC51244 | 334 | | 52 | |
| Fibronectin [Precursor] | S14428 | 275 | | 44 | |
| Laminin alpha-1 chain [precursor]* | MMMSA | 347 | | 40 | |
| Laminin alpha-4 chain [precursor] | LMA4 HUMAN | 204 | | 52 | |
| Laminin alpha-5 chain [precursor] | LMA5_MOUSE | | | | |
| Laminin, gamma 1 | Q5VYE7_HUMAN | | | | 30 |
| Laminin, gamma-3 [precursor]* | AAD36991 | 177 | | 38 | |
| Laminin M | I54245 | 16 | 43 | | |
| Polydom protein [Precursor] | Q9ES77 | 401 | | 41 | |
| Proteoglycan link protein | A29165 | 11 | 35 | | |
| Tenascin-X | T42629 | 454 | | 41 | |
| Thrombospondin 1 | Q80YQ1 | 133 | | 39 | |
| Vitronectin | Q2Y097_9CARN | 7 | 32 | | |
| Membrane | | | | | |
| FGF receptor [Fragment] | C44775 | 3 | 24 | | |
| IGF-II mRNA-binding protein 2 | AAD31596 | 66 | 34 | | |
| IGF-II receptor | Q95M19 | 263 | | 41 | 32 |
| JAK1 protein | AAA36527 | 133 | | 40 | |
| JAK2 protein | Q7TQD0 | 132 | | 49 | |
| Mast/stem cell growth factor receptor | AAA37420 | 2 | 43 | | |
| [Precursor] | | | | | |
| Membrane-type matrix metalloproteinase-1 | Q9XSP0 | 66 | | 41 | |
| Nuclear | | | | | |
| Cell proliferation antigen Ki-67 | T30249 | 325 | | 40 | |
| p53 tetramerization domain* | 1AIE | 3 | 41 | | |
| MAP kinase kinase 7* | Q8BSP1 | 47 | | 40 | |
| MAPK/ERK kinase kinase 4 | T03022 | 183 | | 47 | |
| Protein inhibitor of activated STAT2 | AAF12825 | 64 | 40 | | |
| T-box transcription factor TBX20 | CAC04520 | 33 | 25 | | |
| Tral homolog | TRAP_MOUSE | 294 | | 39 | |
| Ubiquitin carboxyl-terminal hydrolase 43 | Q8N2C5 | 69 | | 59 | |
| Probable E3 ubiquitin-protein ligase | O75592 | 518 | | 49 | |
| MYCBP2 | | | | | |
| Cytoplasmic | | | | | |
| Growth factor receptor-bound protein 14 | AAH53559 | 60 | | | 23 |
| Peroxiredoxin-1 | BAB27120 | 22 | 39 | | |
| Phospholipase C-epsilon* | Q8K4S1 | 258 | | 42 | |
| Casein kinase I isoform alpha | Q5U46 | 37 | | | 25 |
| Secreted | | | | | |
| Interleukin-1 receptor antagonist [Precursor] | AAO24703 | 20 | 37 | | |
| Interleukin-8 (Fragment) | Q6LAA1_CANFA | 7 | 37 | | |
| Matrix-remodelling-associated protein 5 | Q9NR99 | 314 | | 39 | 23 |
| [Precursor] | | | | | |
| Protein Wnt-2b [Precursor] | AAC25397 | 45 | | 41 | |
| Secreted frizzled-related protein 2 * | Q9BG86_RABIT | 33 | 36 | | |
| Suppression of Tumorigenicity 5 | Q924W7 | | | | |
| Serum Derived | | | | | |
| Alpha-2-HS-glycoprotein [Precursor] | S22394 | 39 | | 72 | |
| Serotransferrin [Precursor] | AAA96735 | 79 | | 55 | |
| Serum albumin [Precursor] | AAN17824 | 71 | | 452 | |
| Differentiation and growth factor | | | | | |
| Bone morphogenetic protein 15 | Q8MII6_BOVIN | 12 | 37 | | |
| Follistatin-related protein 1 [Precursor] | S38251 | 35 | | 64 | |
| Hepatocyte growth factor [Precursor] | BAA01065 | 84 | 45 | | |
| IGF-1 [Precursor] | CAA01955 | 13 | 59 | | |
| IGF-II protein (Fragment) | CAA04657 | 8 | 34 | | |
| Platelet-derived growth factor B chain | AAH53430 | 27 | 47 | | |
| [Precursor] | | | | | |
| Pro-epidermal growth factor [Precursor] * | CAA24116 | 4 | 22 | | |
| TGF beta 2 (Fragment) | Q9MYZ1_CAPHI | 10 | 32 | | |
| TGF-beta-induced protein ig-h3 [Precursor] * | AAB88697 | 4 | 22 | | |

TABLE 2
Proteins identified from feeder:hES cell conditioned media using 4000
MALDI-TOF-TOF system, QTRAP MS/MS and, LC-MALDI.

| | | | MALDI | QTRAP | LC- |
|---|---|---|---|---|---|
| | | | MS-MS | MS-MS | MALDI |
| | Accession | MW | Protein | Mowse | Ion |
| Protein | Number | (kDa) | score | Score | Score |
| Extra Cellular Matrix | | | | | |
| Collagen alpha-2(I) chain [Precursor] | AAC64485 | 129 | | 50 | |
| Collagen alpha 1(IV) chain [Precursor] | CGHU4B | 161 | | 38 | |
| Collagen alpha-6(IV) chain [Precursor] | BAA04809 | 163 | | 41 | |
| Collagen alpha-1(V) chain [Precursor] | BAA14323 | 184 | | 41 | |
| Collagen alpha-2(V) chain [Precursor] | Q7TMS0 | 145 | | 41 | |
| Collagen alpha-1(XI) chain [Precursor] | BAA07367 | 181 | | 63 | |
| Collagen alpha-1(XII) chain [Precursor] | AAC51244 | 334 | | 42 | |
| Collagen alpha-1(XV) chain [Precursor] | Q9EQD9 | 140 | | 38 | |
| Laminin alpha-2 chain [Precursor] | S53868 | 351 | | 42 | |
| Laminin subunit alpha-5 [Precursor] * | LMA5_MOUSE | 416 | | 43 | |
| Tenascin X | T09070 | 442 | | 39 | |
| Versican core protein [Precursor]* | T42389 | 371 | | 43 | |
| Membrane | | | | | |
| Cadherin-20 [Precursor] | AAG23739 | 89 | | 39 | |
| Collagen alpha-2(VI) chain [Precursor] * | AAB20836 | 33 | | 44 | |
| Catenin alpha-2* | I49499 | 101 | | 39 | |
| Insulin receptor [Precursor] | AAB61414 | 1 | 20 | | |
| Myelin-oligodendrocyte glycoprotein | Q29ZP9_CALJA | 5 | 17 | | |
| [Precursor] | | | | | |
| Tensin-1 * | Q9HBL0 | 186 | | 41 | |
| Tumour-associated calcium signal | CAA32870 | 35 | 26 | | |
| transducer 1 [Precursor] | | | | | |
| Zeta-sarcoglycan | AAK21962 | 33 | 55 | | |
| Nuclear | | | | | |
| Cell proliferation antigen Ki-67 | T30249 | 325 | | 42 | |
| E3 SUMO | AAC41758 | 362 | | 65 | |
| Fos-related antigen 2 | CAA58804 | 3 | 20 | | |
| Myc-binding protein 2 | O75592 | 518 | | 43 | |
| Mitogen-activated protein kinase 14 | AAC50329 | 34 | 44 | | |
| Progesterone receptor | Q9GLW0 | 99 | | 50 | |
| T-box transcription factor TBX3 | BAC34999 | 79 | 46 | | |
| TGF-beta-inducible nuclear protein 1 | BAB31689 | 6 | 49 | | |
| Transcription factor Dp-2 | TDP2_HUMAN | 49 | | 38 | |
| E3 ubiquitin-protein ligase UHRF1 | O7TPK1 | 94730 | | 39 | |
| Cytoplasm | | | | | |
| Casein kinase I isoform alpha | Q9GLY1 | 37 | | 43 | |
| Dishevelled | DVL3_HUMAN | 78 | | 40 | |
| MAP kinase kinase kinase 4 * | T03022 | 183 | | 42 | 23 |
| Peroxiredoxin | AAH68135 | 147 | | 49 | |
| Protein deltex-4 | AAH58647 | 67 | | 48 | |
| Triple functional domain protein | AAC34245 | 326 | | 40 | |
| Secreted | | | | | |
| Collagenase 3 [Precursor] | AAC24596 | 45 | | | 22 |
| Follistatin-related protein 1 [Precursor] | S38251 | 35 | | 47 | |
| Galanin-like peptide [Precursor] | AAF19724 | 12 | 44 | | |
| Interleukin-2 [Precursor] | CAA42722 | 15 | 47 | | |
| Interleukin-4 [Precursor] | CAA28874 | 3 | 38 | | |
| Interleukin-13 | Q4VB53 | 9 | 40 | | |
| Mealloproteinase-disintegrin domain | Q71U12_MOUSE | 74 | 48 | | |
| containing protein | | | | | |
| Prostaglandin-H2 D-isomerase [Precursor] * | BAA21769 | 21 | | | 23 |
| Suppression of tumorigenicity 5 | AAH36655 | 127 | 44 | | |
| Serum Derived | | | | | |
| Serotransferrin [Precursor] | AAA96735 | 79 | | 94 | |
| Serum albumin [Precursor] | AAN17824 | 71 | | 452 | |

TABLE 3
Proteins identified from feeder cell conditioned media using
LC/ESI/MS and LC-MALDI.

| | | | | Ion |
|---|---|---|---|---|
| | | | Total Ion | Score |
| | Accession | MW | Score | LC- |
| Protein | Number | (kDa) | (LC/ESI/MS) | MALDI |
| Extra-Cellular Matrix | | | | |
| Cartilage intermediate layer protein 1 | O75339 | 135 | 42 | |
| Collagen type 1, alpha-2 | P08123 | 130 | 40 | |
| Collagen type 4, alpha-4 | Q9QZR9 | 166 | 63 | |
| Collagen type 5, alpha-1 | P20908 | 184 | 50 | |
| Collagen type 6, alpha-3 | P12111 | 345 | 44 | |
| Collagen type 7 | Q63870 | 296 | 52 | 19 |
| Collagen type 19, alpha-1 | Q14993 | 116 | 48 | |
| Collagen, type 27, alpha-1 | Q8IZC6 | 187 | 40 | |
| Fibronectin precursor | P11276 | 276 | | 31 |
| Laminin subunit alpha-5 | Q61001 | 416 | 40 | 18 |
| Stretch-responsive fibronectin protein type 3 | Q70X91 | 399 | 41 | |
| Tenascin-X | O18977 | 454 | 38 | |
| Growth Factors | | | | |
| Insulin-like growth factor-I | Q13429 | 15 | | 143 |
| Insulin-like growth factor-II | P09535 | 20 | | 25 |
| Miscellaneous | | | | |
| CaM Kinase ID | Q8IU85 | 43 | | 21 |
| Catalase | P04040 | 60 | 38 | |
| Complement C4 [Precursor] | AAN72415 | 193 | 51 | |
| Discs large homolog 5 | Q8TDM6 | 203 | 43 | |
| Fucosyltransferase 8 | Q543F5 | 67 | 41 | |
| Metastasis suppressor protein 1 | Q8R1S4 | 74 | | 28 |
| Myosin-9 | P35579 | 146 | 44 | |
| Neuronal apoptosis inhibitory protein 5 | Q8BG68 | 162 | | 23 |
| Neutral alpha-glucosidase C type 3 | Q8TET4 | 105 | | 28 |
| Peroxiredoxin 1 | Q9BGI4 | 22 | 42 | |
| Plectin-1 | Q9QXS1 | 535 | 40 | |
| Poly [ADP-ribose] polymerase 14 | Q460N5 | 172 | 48 | |
| Transglutaminase y | Q6YCI4 | 80 | 40 | |
| Tuberin | CAA56563 | 276 | 41 | |
| Tyrosine-protein phosphatase non- | Q64727 | 117 | 38 | |
| receptor type 13 | | | | |
| Uncharacterised progenitor cells protein | Q9NZ47 | 9 | | 25 |
| Vinculin | Q64727 | 117 | 38 | |
| Membrane | | | | |
| Activin receptor type-2B | Q13705 | 58 | 39 | |
| EGF-like domain-containing protein 4 | Q7Z7M0 | 265 | 38 | |
| Fat3 | Q8R508 | 505 | 41 | |
| Hepatocyte growth factor receptor | Q9QW10 | 30 | | 21 |
| Insulin-like growth factor 1 receptor | Q60751 | 158 | | 20 |
| Integrin alpha-7 | Q13683 | 130 | 47 | |
| Intercellular adhesion molecule 1 | Q95132 | 60 | 41 | |
| Chondroitin sulfate proteoglycan 4 | Q6UVK1 | 251 | 40 | |
| Mucin-4 | Q8JZM8 | 367 | 44 | |
| Neurexin-2-alpha | Q9P2S2 | 180 | 38 | |
| Protein patched homolog 2 | O35595 | 130 | | 24 |
| Serine/threonine-protein kinase MARK2 | O08679 | 81 | 48 | |
| Tumour-associated hydroquinone oxidase | Q16206 | 71 | 48 | |
| Ubiquitin thioesterase | T30850 | 293 | 49 | |
| Nuclear | | | | |
| Antigen KI-67 | P46013 | 321 | 55 | 34 |
| PPAR-binding protein | Q925J9 | 105 | 40 | |
| Scapinin | Q8BYK5 | 63 | 38 | |
| Sentrin-specific protease 2 | Q91ZX6 | 67 | | 22 |
| SON protein | P18583 | 260 | 41 | |
| STAT5a | Q3UZ79 | 32 | | 19 |
| Telomerase-binding protein EST1A | P61406 | 162 | 38 | |
| Tra1 homolog | Q80YV3 | 294 | 48 | |
| Zinc finger protein HRX | P55200 | 425 | 46 | |
| Zinc finger protein spalt-3 [Fragment] | Q9EPW7 | 136 | 40 | |
| Secreted | | | | |
| Alpha-fetoprotein | P49066 | 68 | | 78 |
| Insulin | P01317 | 11 | | 22 |
| Kininogen | P01044 | 69 | | 61 |
| Latent-transforming growth factor beta- | Q14767 | 204 | 39 | |
| binding protein 2 | | | | |
| Transferrin | P02787 | 79 | | 64 |
| Serum-Derived | | | | |
| Bovine Serum Albumin | AAN17824 | 71 | 198 | 524 |
| Fetuin | S22394 | 39 | 147 | 124 |
| Hemiferrin | Q64599 | 25 | 91 | |

TABLE 4
Proteins identified from feeder cell:keratinocyte conditioned media
using LC/ESI/MS and LC-MALDI.

| | | | | Ion |
|---|---|---|---|---|
| | | | Total Ion | Score |
| | Accession | MW | Score | LC- |
| Protein | Number | (kDa) | (LC/ESI/MS) | MALDI |
| Extra-Cellular Matrix | | | | |
| Cartilage intermediate layer protein 1 | O75339 | 135 | 42 | |
| Collagen type 1, alpha-2 | P08123 | 130 | 40 | |
| Collagen type 2 alpha-1 | P02458 | 142 | 40 | |
| Collagen type 4, alpha-1 | Q9QZR9 | 166 | 63 | |
| Collagen type 4, alpha-3 | Q9QZS0 | 163 | 39 | |
| Collagen type 7 | P12111 | 345 | 44 | |
| Collagen type 7, alpha-1 | Q63870 | 295 | 56 | |
| Collagen type 11, alpha-2 | P13942 | 172 | 54 | |
| Collagen type 12, alpha-1 | Q99715 | 334 | 55 | |
| Collagen, type 27, alpha-1 | Q8IZC6 | 187 | 40 | 25 |
| Fibronectin 1 | Q3UHL6 | 260 | | 22 |
| Hypothetical fibronectin type III | Q8BKM5 | 82 | 38 | |
| Lamb3 protein | Q91V90 | 132 | 41 | |
| Laminin subunit alpha-1 | CAA41418 | 297 | 69 | |
| Laminin subunit alpha-2 | Q59H37 | 204 | | 27 |
| Laminin subunit alpha-5 | Q61001 | 416 | 40 | |
| Laminin alpha 3b chain | Q76E14 | 376 | 61 | |
| Laminin subunit beta-2 [Precursor] | Q61292 | 203 | 43 | |
| Laminin subunit gamma-3 [Precursor] | Q9Y6N6 | 177 | 39 | |
| Laminin 5 | WO0066731 | 132 | 37 | |
| Stretch-responsive fibronectin protein type 3 | Q70X91 | 399 | 41 | |
| Tenascin-X | O18977 | 454 | 38 | |
| Cytoplasm | | | | |
| Liprin-alpha-2 | Q8BSS9 | 143 | 63 | |
| Liprin-alpha-3 | O75145 | 133 | 40 | |
| Serine/threonine-protein kinase TAO1 | Q7L7X3 | 116 | 42 | |
| Growth Factors | | | | |
| Transforming growth factor alpha | P01135 | 18 | | 31 |
| Miscellaneous | | | | |
| Actin alpha 2 | P62736 | 42 | 81 | |
| Actin, beta [Fragment] | Q96HG5 | 41 | 78 | |
| Ankyrin-3 | Q12955 | 482 | 37 | |
| Carbamoyl-phosphate synthetase I | P31327 | 165 | 38 | |
| Catalase | P04040 | 60 | 38 | |
| CDNA FLJ11753 fis, clone | Q9HAE5 | 32 | 37 | |
| HEMBA1005583 | | | | |
| Complement C4 [Precursor] | AAN72415 | 193 | 51 | |
| Discs large homolog 5 | Q8TDM6 | 203 | 43 | |
| Dystrophin | P11531 | 427 | 48 | |
| Exostosin-1 | Q16394 | 87 | 48 | |
| Fucosyltransferase 8 | Q543F5 | 67 | 41 | |
| Granulocyte inhibitory protein II homolog | Q9UD48 | 2 | | 31 |
| Hypothetical protein | Q8C7W2 | 55 | 40 | |
| Kinesin-like protein KIF13A | Q9H1H9 | 200 | 46 | |
| Myosin-9 | P35579 | 146 | 44 | |
| myosin-IXb | Q14788 | 230 | 45 | |
| Myosin-XVIIIa | Q9JMH9 | 117 | 42 | |
| Neuron navigator 3 | Q8NFW7 | 245 | 58 | |
| Peroxiredoxin 1 | Q9BGI4 | 22 | 42 | |
| Plectin-1 | Q9QXS1 | 535 | 40 | |
| Poly [ADP-ribose] polymerase 14 | Q460N5 | 172 | 48 | |
| Protein diaphanous homolog 2 | O70566 | 125 | 46 | |
| Protein disulfide-isomerase | P04785 | 30 | 37 | |
| Protein piccolo | Q9Y6V0 | 568 | 59 | |
| Sacsin | Q9NZJ4 | 441 | 39 | |
| Serine protease inhibitor EIC | Q8K3Y1 | 42 | 41 | |
| Transglutaminase y | Q6YCI4 | 80 | 40 | |
| Tuberin | CAA56563 | 276 | 41 | |
| Tyrosine-protein phosphatase non- | Q64727 | 117 | 38 | |
| receptor type 13 | | | | |
| Ubiquitin specific protease 1 | Q8BJQ2 | 88 | 58 | |
| Membrane | | | | |
| Acetyl-CoA carboxylase 2 | O00763 | 281 | 39 | |
| Cadherin EGF LAG seven-pass G-type | Q91ZI0 | 363 | 40 | |
| receptor 3 | | | | |
| Cation-independent mannose-6- | P11717 | 281 | 41 | |
| phosphate receptor | | | | |
| Chondroitin sulfate proteoglycan 4 | Q6UVK1 | 251 | 40 | |
| Cytokeratin-1 | P04264 | 66 | 68 | |
| Cytokeratin-9 | P35527 | 62 | 127 | |
| EGF-like domain-containing protein 4 | Q7Z7M0 | 265 | 38 | |
| EMR1 hormone receptor | Q14246 | 101 | 40 | |
| Fat3 | Q8R508 | 505 | 41 | |
| Integrin beta-4 | P16144 | 211 | 43 | |
| Integrin alpha-7 | Q13683 | 130 | 47 | |
| Intercellular adhesion molecule 1 | Q95132 | 60 | 41 | |
| Mucin-4 | Q8JZM8 | 367 | 44 | |
| Mucin-16 | Q8WXI7 | 747 | 49 | |
| Neurexin-2-alpha | Q9P2S2 | 180 | 38 | |
| RIM ABC transporter | P78363 | 258 | 58 | |
| Serine/threonine-protein kinase MARK2 | O08679 | 81 | 48 | |
| Talin-1 | Q9Y490 | 273 | 43 | |
| Talin-2 | Q9Y4G6 | 274 | 40 | |
| Tumor-associated hydroquinone oxidase | Q16206 | 71 | 48 | |
| Ubiquitin thioesterase | T30850 | 293 | 49 | |
| Nuclear | | | | |
| DNA-binding protein SMUBP-2 | Q60560 | 109 | 43 | |
| Antigen KI-67 | CAA46520 | 321 | 42 | |
| Lipin-3 | Q7TNN8 | 95 | 38 | |
| Nesprin-2 | AAL33548 | 801 | 53 | |
| Nef-associated factor 1 | Q15025 | 35 | 46 | |
| NFX1-type zinc finger-containing protein 1 | Q9P2E3 | 225 | 46 | |
| Periaxin | AAK19279 | 155 | 52 | |
| PPAR-binding protein | Q925J9 | 105 | 40 | |
| Putative rRNA methyltransferase 3 | Q9DBE9 | 95 | 48 | |
| Scapinin | Q8BYK5 | 63 | 38 | |
| SET-binding factor 1 | O95248 | 210 | 41 | |
| SON protein | P18583 | 233 | 42 | |
| Telomerase-binding protein EST1A | P61406 | 161 | 49 | |
| Tra1 homolog | Q80YV3 | 294 | 48 | |
| Transcription factor 7-like 2 | Q924A0 | 52 | | 34 |
| TTF-I-interacting protein 5 | Q9UIF9 | 210 | 57 | |
| Zinc finger protein HRX | P55200 | 425 | 46 | |
| Zinc finger protein spalt-3 [Fragment] | Q9EPW7 | 136 | 40 | |
| Zinc finger protein 40 | P15822 | 299 | 57 | |
| Secreted | | | | |
| Apolipoprotein A-II | P81644 | 8 | 56 | |
| Follistatin-related protein 5 | Q8BFR2 | 95 | | 27 |
| Latent-transforming growth factor beta- | Q28019 | 208 | 38 | |
| binding prot-2 | | | | |
| Matrix-remodeling-associated protein 5 | Q9NR99 | 314 | 39 | |
| Nidogen | P10493 | 139 | | 26 |
| Platelet glycoprotein V | Q9QZU3 | 64 | 39 | |
| Proteoglycan-4 [Precursor] | Q9JM99 | 117 | 37 | |
| SCO-spondin [Precursor] | P98167 | 575 | 38 | |
| Transferrin | P02787 | 79 | | 64 |
| Serum-Derived | | | | |
| Bovine Serum Albumin | AAN17824 | 71 | 198 | 335 |
| Fetuin | S22394 | 39 | 147 | 126 |
| Hemiferrin | A39684 | 24 | 50 | |
| Human Serum Albumin | CAA23753 | 71 | 64 | |

TABLE 5
Differences in expression of protein species found in the feeder cell
and the feeder cell:hES/Keratinocyte conditioned media

| | Score |
|---|---|
| | Ion (I) |
| | Protein |
| | (P) |
| Feeder Cell Alone | |
| Extra-cellular Matrix | |
| Collagen I | I-40 |
| Collagen IV | I-63 |
| Collagen V | I-50 |
| Collagen VI | I-44 |
| Collagen VII | I-52 |
| Fibronectin I | I-31 |
| Laminin V | I-40 |
| Growth Factors and Cytokines | |
| BMP 1 | P-25 |
| BMP15 | P-37 |
| bFGF | P-32 |
| FGF homologous factor 3 | P-20 |
| Human Growth hormone | P-34 |
| Insulin | I-22 |
| Insulin-like growth factor 1 | I-143 |
| Insulin-like growth factor 2 | I-25 |
| TGF alpha | P-14 |
| TGF beta 2 | P-34 |
| VEGF | P-20 |
| Interleukin 1 beta | P-39 |
| interleukin-8 | P-32 |
| Interleukin 10 | P-32 |
| Isoform of interleukin 15 | P-25 |
| Leukemia inhibitory factor | P-33 |
| Hepatocyte growth factor | P-45 |
| Secreted | |
| Megakaryocyte-CSF | P-22 |
| Wnt-2b | I-41 |
| Secreted frizzled-related protein 2 | P-36 |
| Follistatin-related protein 1 | I-64 |
| Wnt-12 | P-30 |
| Intracellular | |
| Telomerase-binding protein EST1A | I-38 |
| Tra1 homolog | I-48 |
| Feeder Cell:hES/Keratinocyte | |
| Extra-cellular Matrix | |
| Collagen I | I-40 |
| Collagen IV | I-63 |
| Collagen VII | I-44 |
| Fibronectin I | I-22 |
| Fibronectin III | I-38 |
| Laminin I | I-69 |
| Laminin III | I-61 |
| Laminin V | I-37 |
| Growth Factors and Cytokines | |
| FGF-2 associated protein 3 | P-36 |
| NGF homolog 1 | P-40 |
| PC cell-derived growth factor | P-36 |
| PDGF bb | P-16 |
| TGF alpha | I-31 |
| TGF beta I | P-18 |
| VEGF | P-20 |
| Interleukin 1 alpha | P-21 |
| Interleukin-2 | P-47 |
| Interleukin 4 | P-20 |
| Interleukin 10 | P-21 |
| Interleukin-6 | P-37 |
| Shorter isoform of interleukin 15 | P-19 |
| PDGF-inducible JE glycoprotein | P-43 |
| HGF | P-45 |
| Secreted | |
| Follistatin-related protein 5 | I-27 |
| growth inhibitory factor | P-15 |
| Growth differentiation factor 9 | P-31 |
| Intracellular | |
| Telomerasereverse transcriptase | P-31 |
| Telomerase-binding protein EST1A | I-49 |
| Tra1 homolog | I-48 |

1. A cell culture medium, comprising:
(i) a synthetic chimeric protein comprising an insulin-like growth factor (IGF) amino acid sequence and a vitronectin (VN) amino acid sequence;
(ii) one or more isolated feeder cell-replacement factors selected from the group consisting of human growth hormone (hGH), bone morphogenic protein 15 (BMP-15), growth differentiation factor 9 (GDF-9), megakaryocyte colony-stimulating factor, secreted frizzled-related protein 2, Wnt-2b, Wnt-12, growth inhibitory factor, fetuin, human serum albumin (HSA), hepatocyte growth factor (HGF), transforming growth factor-α (TGF-α), TGF-β, nerve growth factor, platelet derived growth factor-β (PDGF-β), PC-derived growth factor (progranulin), interleukin (IL)-1, IL-2, IL-4, IL-6, IL-8, IL-10, IL-13 and Activin-A; and
(iii) an absence of serum or a substantially reduced amount of serum which in the absence of an IGF would not support cell growth.

2. The cell culture medium of claim 1, wherein the one or more isolated feeder cell-replacement factors are selected from the group consisting of hGH, BMP-15, GDP-9, megakaryocyte colony-stimulating factor, secreted frizzled-related protein 2, Wnt-2b, Wnt-12, growth inhibitory factor and Activin-A.

3. The cell culture medium claim 2, wherein the one or more isolated feeder cell-replacement factors is Activin-A.

4. The cell culture medium of claim 1, wherein the cell culture medium further comprises one or more additional biologically active proteins selected from the group consisting of basic fibroblast growth factor (bFGF), epidermal growth factor (EGF), IGF-I, IGF-II and a laminin.

5. The cell culture medium of claim 4, wherein the one or more additional biologically active proteins are selected from bFGF and a laminin.

6. The cell culture medium of claim 1, wherein the IGF amino acid sequence is an IGF-I amino acid sequence or an IGF-II amino acid sequence.

7. The cell culture medium of claim 6, wherein the IGF amino acid sequence is an IGF-I amino acid sequence.

8. The cell culture medium of claim 1, wherein the VN amino acid sequence is amino acid residues 1 to 64 of mature VN.

9. The cell culture medium of claim 1, wherein the synthetic chimeric protein further comprises a linker sequence of one or more glycine residues and one or more serine residues.

10. The cell culture medium of claim 9, wherein the linker sequence is (Gly₄Ser)₄.

11. The cell culture medium of claim 1, which further comprises an isolated IGF-containing complex wherein the IGF is selected from IGF-I and IGF-II.

12. The cell culture medium of claim 11, which further comprises VN when IGF-II is present in the isolated IGF-containing complex.

13. The cell culture medium of claim 11, which further comprises an IGFBP and VN when IGF-I is present in the isolated IGF-containing complex.

14. The cell culture medium of claim 13, wherein the IGFBP is selected from the group consisting of IGFBP-1, IGFBP-2, IGFBP-3, IGFBP-4, IGFBP-5 and IGFBP-6.

15. The cell culture medium of claim 14, wherein the IGFBP is IGFBP-3 or IGFBP-5.

16. The cell culture medium of claim 1, wherein the or each feeder cell-replacement factor has a final concentration of between 0.1 ng/ml and 50 μg/ml.

17. The cell culture medium of claim 16, wherein the or each feeder cell-replacement factor has a final concentration of between about 5 ng/ml and 1500 ng/ml.

18. The cell culture medium of claim 17, wherein the or each feeder cell-replacement factor has a final concentration of between 25 ng/ml and 1000 ng/ml.

19. The cell culture medium of claim 18, wherein the or each feeder cell-replacement factor has a final concentration of between 150 ng/ml and 600 ng/ml.

20. An embryonic stem cell culture medium comprising between about 250 ng/ml and 1000 ng/ml of a synthetic chimeric protein comprising an IGF amino acid sequence and a VN amino acid sequence, between about 50 ng/ml and 100 ng/mlof bFGF, between about 25 ng/ml and 50 ng/ml of Activin-A and between about 10 μg/ml and 50 μg/ml of a laminin.

21. The embryonic stem cell culture medium of claim 20 comprising about 1000 ng/ml of the synthetic chimeric protein comprising an IGF amino acid sequence and a VN amino acid sequence, about 100 ng/ml of bFGF, about 35 ng/ml Activin-A and about 40 μg/ml of the laminin.

22. The embryonic stem cell culture medium of claim 21, wherein the IGF amino acid sequence is an IGF-I amino acid sequence or an IGF-II amino acid sequence.

23. The embryonic stem cell culture medium of claim 22, wherein the IGF amino acid sequence is an IGF-I amino acid sequence.

24. The embryonic stem cell culture medium of claim 21, wherein the VN amino acid sequence is amino acid residues 1 to 64 of mature VN.

25. A cell culture system comprising a culture vessel and the cell culture medium of claim 1.

26. A method of cell culture including the step of culturing one or more cells in the cell culture medium claim 1.

27. The method of claim 26, wherein the one or more cells are a feeder dependent cell type.

28. The method of claim 27, wherein the one or more cells are human embryonic stem cells.

29. The method of claim 27, wherein the one or more cells are keratinocytes.

30. A pharmaceutical composition comprising one or more cells produced according to the method of claim 26, together with a pharmaceutically acceptable carrier, diluent or exicipient.

31. The pharmaceutical composition of claim 30 comprising one or more cells selected from the group consisting of keratinocytes, human embryonic stem cells and keratinocyte progenitor cells.

32. The pharmaceutical composition of claim 31, wherein the one or more cells are human embryonic stem cells.

33. The pharmaceutical composition of claim 31, wherein the one or more cells are keratinocytes.

34. A method of delivering one or more cells cultured according to the method of claim 30, to an individual to thereby facilitate renewal and/or proliferation of one or more cells in said individual.

35. A cell culture system comprising the embryonic stem cell culture medium of claim 20.

36. A method of cell culture including the embryonic stem cell culture medium of claim 20.

37. A method of cell culture including the culture system of claim 25.

38. A method of cell culture including the step of culturing one or more cells in the cell culture medium of the culture system of claim 35.


SEQUENCE LISTING (6 sequences)

SEQ ID NO: 1 - DNA, 25 bp, Homo sapiens
cttgctgcagaagtgggtggaggaa

SEQ ID NO: 2 - DNA, 21 bp, Homo sapiens
ctgcagtgtgggtttcgggca

SEQ ID NO: 3 - DNA, 20 bp, Homo sapiens
tcagaagctcaacaccaacg

SEQ ID NO: 4 - DNA, 20 bp, Homo sapiens
ttgtacgtcttggagagggc

SEQ ID NO: 5 - DNA, 20 bp, Homo sapiens
ttcggaactgaggccatgat

SEQ ID NO: 6 - DNA, 21 bp, Homo sapiens
cgaacctccgactttcgttct